(19) United States
(12) Patent Application Publication
(10) Pub. No.: US 2026/0022325 A1
(43) Pub. Date: Jan. 22, 2026

(54) CELL MANUFACTURING ON AN AUTONOMOUS PLATFORM

(71) Applicant: Cellino Biotech, Inc., Cambridge, MA (US)

(72) Inventors: Marinna Madrid, Cambridge, MA (US); Bar Makovoz, Cambridge, MA (US); Matthias Wagner, Cambridge, MA (US); Alexander Stange, Somerville, MA (US); Christopher Harrison, Cambridge, MA (US); Catherine Pilsmaker, Arlington, MA (US); Kien Tran, Cambridge, MA (US); Ana D'Alessio, Cambridge, MA (US)

(21) Appl. No.: 19/273,771

(22) Filed: Jul. 18, 2025

Related U.S. Application Data
(60) Provisional application No. 63/672,831, filed on Jul. 18, 2024.
(60) Provisional application No. 63/689,112, filed on Aug. 30, 2024.
(60) Provisional application No. 63/689,115, filed on Aug. 30, 2024.
(60) Provisional application No. 63/692,355, filed on Sep. 9, 2024.
(60) Provisional application No. 63/709,763, filed on Oct. 21, 2024.
(60) Provisional application No. 63/742,210, filed on Jan. 6, 2025.

(51) Int. Cl. C12M 1/36; C12M 1/00; C12M 1/34; G06T 7/00

(52) U.S. Cl. CPC C12M 41/48; C12M 41/36; C12M 47/02; G06T 7/0016; G06T 2207/10016; G06T 2207/30024

(57) ABSTRACT

A method for cell culture expansion and maintenance, comprising seeding, in a first cell culture cassette, a plurality of cell colonies adhered to a first surface of the first cell culture cassette; capturing, by an optical engine, time-series images of the plurality of cell colonies as the plurality of cell colonies expand; determining, by a platform manager, a subset of cell colonies to remove from the first surface based on the time-series images; removing, by the optical engine, the subset of cell colonies from the first surface; washing, by a fluid management system, the removed subset of cell colonies from the first cell culture cassette; repeating steps for one or more iterations; harvesting a set of remaining cell colonies from the first cell culture cassette; seeding the set of remaining cell colonies into a second cell culture cassette; repeating steps for additional iterations in the second cell culture cassette.

### CROSS-REFERENCE TO RELATED APPLICATIONS

This application claims the benefit of priority to U.S. Provisional Application No. 63/672,831, filed Jul. 18, 2024, U.S. Provisional Application No. 63/689,112, filed Aug. 30, 2024, U.S. Provisional Application No. 63/689,115, filed Aug. 30, 2024, U.S. Provisional Application No. 63/692,355, filed Sep. 9, 2024, U.S. Provisional Application No. 63/709,763, filed Oct. 21, 2024, and U.S. Provisional Application No. 63/742,210, filed Jan. 6, 2025, each of which is incorporated herein by reference in its entirety.

### BACKGROUND

Autologous induced pluripotent stem cell (iPSC)-derived regenerative cell therapies that utilize the patient's own cells offer a promising avenue for addressing a variety of medical conditions. They minimize immune-related complications, and are exceptionally well-suited to meet the needs of an aging and increasingly diverse patient population.

Despite significant medical advantages to patients, the current artisanal cell manufacturing methods of generating autologous iPSC-derived therapies face substantial challenges in terms of scalability. These issues pertain to cost-effectiveness and volume production, rendering autologous cell therapies economically unfeasible. Currently, iPSCs are manufactured in high-grade clean rooms by scientists over 10-12 weeks. Somatic cells, like skin fibroblasts or blood cells, are isolated and then cultured in dishes. Scientists introduce reprogramming factors, such as Oct4, Sox2, Klf4, and c-Myc, to these cells, often using Sendai viral vectors. Once pluripotent colonies form, researchers carefully pick and transfer these colonies under a microscope to new culture dishes for an extensive stabilization and vector clearing phase, followed by expansion and harvest. Resulting iPSCs are subjected to quality control (QC) release testing and cryopreserved for long-term storage.

This manual process is time-consuming and expensive, and in its current form cell therapies would only be available to those who are able to pay the significant cost involved. In addition, many cell therapy providers would have a difficult time even getting regulatory approval because of scaling issues in the late clinical trial stages. In addition, manual processes may be subject to many delays due to quality control issues, staffing issues, and other problems. Thus, there is a need in the art for scalable, automated or semi-automated solutions to enable wide-scale and efficient adoption of regenerative cell therapies, and these solutions must address the myriad technical challenges involved in automating a multi-month process performed by skilled technicians.

### BRIEF SUMMARY

In an exemplary embodiment, a method of cell culture management is provided. The method includes seeding a plurality of cell colonies adhered to a first surface of a first cell culture cassette. An optical engine captures time-series images of the cell colonies as they expand. A platform manager determines a subset of cell colonies to remove from the first surface based on the time-series images. The optical engine removes the subset of cell colonies from the first surface, and a fluid management system washes the removed subset of cell colonies from the first cell culture cassette. These steps are repeated for one or more iterations. The optical engine then harvests a set of remaining cell colonies from the first cell culture cassette. The set of remaining cell colonies is seeded into a second cell culture cassette, and the process is repeated for one or more iterations in the second cell culture cassette.

In another exemplary embodiment, the method further includes repeating the seeding and subsequent steps for one or more additional cell culture cassettes.

In a further exemplary embodiment, the method includes harvesting cells for use in a cell therapy after completing the process with the additional cell culture cassettes.

In yet another exemplary embodiment, determining the subset of cell colonies involves applying a machine learning model to the time-series images.

In an additional exemplary embodiment, determining the subset of cell colonies comprises ranking the plurality of cell colonies according to a set of criteria and removing one or more lowest ranked cell colonies.

In another exemplary embodiment, the set of criteria for ranking cell colonies includes parameters corresponding to clonal colony quality.

In a further exemplary embodiment, determining the subset of cell colonies includes removing a portion of a highest ranked cell colony for analysis.

In yet another exemplary embodiment, determining the subset of cell colonies involves removing cell colonies or portions of cell colonies to reduce cell confluence on the first surface.

In an additional exemplary embodiment, determining the subset of cell colonies includes removing cell colonies or portions of cell colonies to shape remaining cell colonies in a pattern.

In another exemplary embodiment, harvesting the set of remaining cell colonies comprises harvesting a single, clonal cell colony.

In a further exemplary embodiment, harvesting the set of remaining cell colonies involves harvesting all cells therein when cell confluence reaches a predetermined threshold.

In yet another exemplary embodiment, the set of remaining cell colonies is transferred into the second cell culture cassette as single cells, cell colonies, or cell sheets.

In an additional exemplary embodiment, the first surface comprises an optical film configured for imaging and optical removal of cells by the optical engine.

In another exemplary embodiment, the process of removing and washing cell colonies is performed over a period of at least 10 days.

In a further exemplary embodiment, determining the subset of cell colonies includes analyzing morphological characteristics of the plurality of cell colonies in the time-series images.

In yet another exemplary embodiment, the morphological characteristics analyzed include at least one of colony size, colony shape, cell density within the colony, or cell arrangement within the colony.

In an additional exemplary embodiment, the optical engine comprises a laser-based cell removal tool configured to selectively remove cells from the first surface.

In another exemplary embodiment, seeding the set of remaining cell colonies into the second cell culture cassette involves dissociating the remaining cell colonies into single cells prior to seeding.

In a further exemplary embodiment, the method includes applying a rejuvenation protocol to the cells in the second cell culture cassette.

In yet another exemplary embodiment, the rejuvenation protocol comprises introducing factors for partial cellular reprogramming.

In an exemplary embodiment, a system for cell culture management is provided. The system includes a first cell culture cassette configured for a plurality of cell colonies to be seeded to and adhere to a first surface thereof. An optical engine is configured to capture time-series images of the cell colonies as they expand. A platform manager is configured to determine a subset of cell colonies to remove from the first surface based on the time-series images, and the optical engine is further configured to remove the subset of cell colonies from the first surface. A fluid management system is configured to wash the removed subset of cell colonies from the first cell culture cassette. The system also includes a second cell culture cassette configured for a set of remaining cell colonies harvested from the first cell culture cassette to be seeded therein.

In another exemplary embodiment, the system is configured to perform the method of cell culture management described in the previous embodiments.

In an exemplary embodiment, a computer-implemented method of cell culture management is provided. The method includes receiving time-series images of a plurality of cell colonies adhered to a first surface of a first cell culture cassette as the cell colonies expand. The method determines a subset of cell colonies to remove from the first surface based on the time-series images. Instructions are sent to an optical engine to remove the subset of cell colonies from the first surface and to a fluid management system to wash the removed subset of cell colonies from the first cell culture cassette. The method also includes sending instructions to the optical engine to harvest a set of remaining cell colonies from the first cell culture cassette.

In another exemplary embodiment, a computer program product for cell culture management is provided. The computer program product includes a computer readable storage medium having program instructions embodied therewith. The program instructions are executable by a processor to cause the processor to perform the computer-implemented method of cell culture management described in the previous embodiment.

### BRIEF DESCRIPTION OF THE DRAWINGS

FIG. 1 is a block diagram illustrating an autonomous cell manufacturing platform in accordance with various implementations.

FIG. 2 is a diagram of an operating environment enabled by pluggable fluidic cassettes in accordance with various implementations.

FIG. 3 is a diagram illustrating another operating environment for performing batched bioprocesses in accordance with various implementations.

FIG. 4 is a diagram illustrating another operating environment for pluggable cassette-based systems in accordance with various implementations.

FIG. 5 is a diagram illustrating another operating environment utilizing shared equipment by multiple cell batches in accordance with various implementations.

FIG. 6 is a diagram illustrating another operating environment supporting aseptic normally-closed pluggable cassette-based systems in accordance with various implementations.

FIG. 7 is a diagram illustrating a method of pre-patterning cell colonies in accordance with various implementations.

FIGS. 8A-V are diagrams illustrating an example series of cell culture process operations in accordance with various implementations.

FIGS. 9A-D are diagrams illustrating an example method of an in-place expansion sequence for a cell colony in accordance with various implementations.

FIGS. 10A-E are diagrams illustrating another example method of an in-place expansion sequence for a cell colony in accordance with various implementations.

FIG. 11 is a diagram illustrating several normally-closed pluggable fluidic cassettes for use in iPSC reprogramming processes in accordance with various implementations.

FIG. 12 is a diagram illustrating an example workflow using pluggable cassettes in iPSC reprogramming in accordance with various implementations.

FIG. 13 is a flow chart illustrating a method of manufacturing brown or beige adipose tissue in a cell manufacturing platform in accordance with various implementations.

FIG. 14 is a flow chart illustrating a method of an example bioprocess for rejuvenation of specific cell types in accordance with various implementations.

FIGS. 15A-B are diagrams illustrating closed cassettes configured to support the growth and processing of epithelial cells in accordance with various implementations.

FIGS. 16A-B are diagrams depicting methods of seeding RPE cells in a closed cassette with density control in accordance with various implementations.

FIG. 17 is a diagram illustrating performing media changes in a closed cassette growing RPEs in accordance with various implementations.

FIG. 18 is a diagram illustrating imaging of a closed cassette containing RPEs in accordance with various implementations.

FIGS. 19A-B are diagrams of a cassette configured to make trans-epithelial electrical resistance measurements of RPE cells in accordance with various implementations.

FIG. 20 is a diagram illustrating harvesting of suspension RPE cells from a cassette in accordance with various implementations.

FIG. 21 is a diagram illustrating harvesting of RPE cell sheets from a cassette in accordance with various implementations.

FIG. 22 is a flow chart illustrating a method of manufacturing retinal pigment epithelial tissue in a cell manufacturing platform in accordance with various implementations.

FIG. 23 is a flow chart illustrating a method of manufacturing dopaminergic cells in a cell manufacturing platform in accordance with various implementations.

These and other features of the present implementations will be understood better by reading the following detailed description, taken together with the figures herein described. The accompanying drawings are not intended to be drawn to scale. For purposes of clarity, not every component may be labeled in every drawing.

### DETAILED DESCRIPTION

The systems and methods disclosed herein include an autonomous cell manufacturing platform for efficient and scalable production of cells (e.g., iPSCs, differentiated cells) for use in cell therapies. The cell manufacturing platform utilizes optical bioprocesses such as optical imaging and optical-based cell culture management to continuously monitor and control cell culture processes without the need for constant human intervention. Cells are cultured, monitored, and managed in compact closed systems, such as closed cassettes, to provide a mobile, sterile cell culture environment while allowing for high multi-patient throughput in the overall system. The manufacturing platform also includes incubation spaces for cells to expand or grow over long periods of time, a fluid management system to inject and remove fluid media from the closed cassettes, and robotic elements to move the closed cassettes around the platform. The manufacturing platform utilizes artificial intelligence (AI) to analyze cell culture images and make determinations about cell interventions (e.g., cell removal, cell harvesting, media changes).

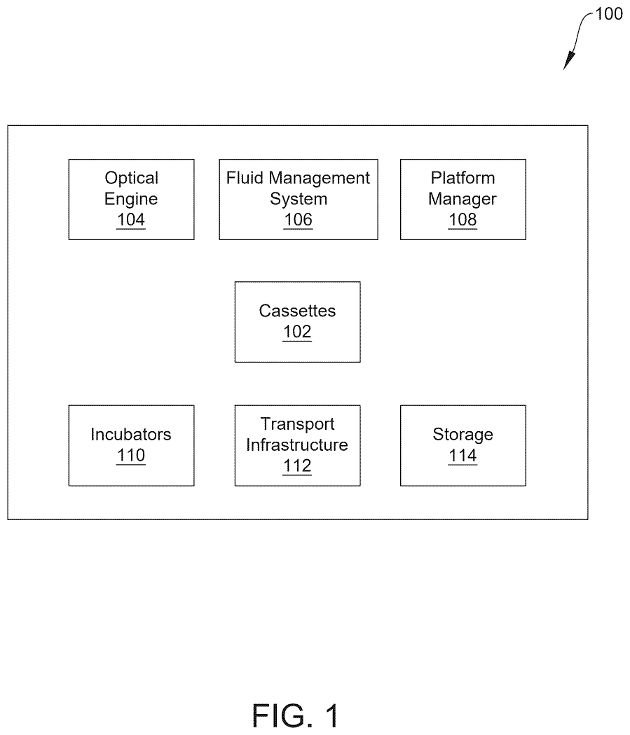

FIG. 1 is a block diagram illustrating an autonomous cell manufacturing platform 100 in accordance with various implementations. The platform 100 enables scalable, autonomous, and efficient production of cells for cell therapies. The platform 100 may provide a controlled, sterile environment for the manufacturing of cells for cell therapies. For example, the platform 100 may be a sealed enclosure with minimal interfaces to external environments and may be sterilized between uses. The platform 100 may be configured to adhere to various regulatory requirements for the production of cell and drug products administrable to patients.

Platform 100 includes a plurality of cassettes 102. Each cassette 102 may support a cell culture. The cell culture may start as source cells that undergo a cell culture process to produce an output cell product. For example, the source cells may be somatic cells that undergo reprogramming and expansion into output iPSC cells. In another example, the source cells may be iPSC cells that undergo differentiation and expansion into differentiated cells for use in cell therapies. Each cassette 102 may be a closed, sterile system that prevents contamination of the cell samples and allows for multi-sample and/or multi-patient processing using shared infrastructure.

Each cassette 102 may include one or more cell culture chambers, which may be closed fluidic chambers for growing cells (e.g., adherent cells). The cell culture chambers may include at least one transparent surface that includes an optical film upon which the cells adhere. The optical film may be flat and permanently attached to the first surface of the cell culture chamber. The optical film may be a multi-layered composition that includes various layers to enable selective light absorption, promote cell adherence, and prevent leaching of materials into the cell culture chamber. The optical film may enable optical-based label-free imaging and optical-based cell manipulation and removal techniques (e.g., using a laser). For example, the optical film may be configured to transmit wavelengths within certain wavelength ranges for imaging applications and at least partially absorb wavelengths in other wavelength ranges for optical removal applications. The cassettes 102 may be in a format that allows for observation of the cell culture at regular intervals. The cassettes 102 may also include various other components to enable autonomous optical processes, transport, and fluid exchange. For example, the cassettes 102 may include fiducials used to align imaging equipment, ports and tubing to enable fluid exchanges, and handles or other mechanical features to enable gripping, rotation, transport, or other physical manipulations of the cassette 102. In some implementations, the cassette 102 may have different designs and configurations for different purposes (e.g., expansion cassette, growth/maintenance cassette, differentiation cassette, harvesting cassette).

The platform 100 also includes an optical engine 104, which is configured to provide optical imaging and cell intervention functionality on the platform 100. Optical-based processes allow cell cultures to be monitored and managed without mechanical means, and thus not breaking the closed, sterile environment of the cassettes 102. The optical engine 104 may be located in a particular location within the platform 100, and cassettes 102 may be moved to the optical engine 104 from a storage location by robotic means for performing imaging and cell management functions. The optical engine 104 may be configured to provide label-free imaging suitable for long-term cell culture observation, although some implementations may include fluorescent imaging capability for immunofluorescent or other labeled images. The optical engine 104 may be configured to collect time-series images of cell cultures in the cassettes, which may be used by machine learning models to analyze cell growth, make predictions on future cell growth, and make determinations about interventions to perform on the cell culture.

The optical engine 104 may also be configured to function as a cell removal tool, or perform other methods of optical-based manipulation of the cell culture (e.g., cell poration, removal of ECM) in the cassettes 102. The optical engine 104 may be configured to target and remove cells at a regional, cluster-specific, and/or cell-specific level. Removal, in this context, may include selective destruction and/or removal of cells or cell regions, and non-destructive operations on cells (including intracellular delivery of compounds into cells or extraction of compounds from cells). The optical engine 104 may also be used to perform cell operations on a cell culture, such as splitting cell colonies into multiple sub-colonies, translating cell colonies across a cell culture surface, reducing confluence, surface area, or density of cell colonies by selective removal, and clonalization of cell colonies by repeated culling of portions of the cell colonies. In some implementations, the platform 100 may include more than one optical engine 104 that is shared by the cassettes 102.

One example implementation of the optical engine 104 is a laser-based system. The optical engine 104 may emit light within a first wavelength range for imaging cells in the cassettes 102. The optical engine 104 may also emit laser pulses within a second wavelength range that are designed to remove cells from the cell growth surface. Removal may be effectuated by, for example, heat transfer of energy from the laser pulses to the cells to dislodge/kill them, or conversion of optical energy into mechanical energy via the formation of microbubbles that kill and/or dislodge the cells.

The platform 100 also includes a fluid management system 106 that is configured to handle the injection and removal of fluids from the cassettes 102. Fluid media includes nutrients necessary for cells to grow, and cells expel waste into the fluid media. Thus, the fluid media must be periodically refreshed for cells to grow and be maintained in a healthy state. The fluid management system 106 may be located in a particular location within the platform 100, and cassettes 102 may be moved to the fluid management system 106 from a storage location by robotic means for performing fluidic exchange functions. The fluid management system 106 may include a receptacle for holding cassettes 102 in place during the fluid exchange. In some implementations, the receptacle may be configured to rotate, translate, or shake/vibrate the cassettes to achieve various fluid manipulation functions. The fluid management system 106 may also include tubing, pipetting, ports, and connectors to connect the cassettes with fluid and waste reservoirs. In some implementations, the fluid management system 106 is configured to aseptically connect to the cassettes to prevent contamination of the cell culture during fluid exchanges. In some implementations, the platform 100 may include more than one fluid management system 106 that is shared by the cassettes 102.

The platform 100 also includes a platform manager 108 configured to monitor and control the other components of the platform 100. The platform manager 108 may be, for example, a combination of on-premises and cloud computing resources that provide data collection, data analysis, and control functions. The platform manager 108 may be configured to gather data from a range of sources, organize the data in a manner that allows it to make predictions of success/quality/functionality of the cell culture, and in many cases do so on a cell-by-cell, cluster-by-cluster, or region-by-region basis. The platform manager 108 may utilize various artificial intelligence and machine learning models to monitor, analyze, and manage cell culture processes. The platform manager 108 may control the optical engine 104 according to cell management algorithms (for example, to maintain a certain cell density, to maintain certain exclusion areas within the cell culture container), in a timed manner (for example, delivering gene-activating or gene-editing compounds to cells at a specific interval), and/or as a result of predictions made by the platform manager 108 (for example, removal of cells predicted not to yield the desired phenotype or optimal level of function).

The platform 100 may also include one or more incubators 110. The incubators 110 may serve as storage locations for the cassettes 102 during cell growth, expansion, or maintenance, when the cassettes 102 are not being transported to the optical engine 104 or the fluid management system 106. The incubators 110 may be maintained at certain temperatures conducive for cell growth. The platform 100 may also include transport infrastructure 112 for moving cassettes 102 within the platform (e.g., from the incubators 110 to the optical engine 104 and back). The transport infrastructure 112 may include, for example, robotic arms that can grasp and move the cassettes, and/or rails that can transport the cassettes 102 from one location to another. The platform 100 may also include storage space 114, which may be used to store consumables within the platform. Such consumables may include, for example, fluids, pipettes, connectors, and other one-time use components. Such storage spaces may be temperature regulated, for example at 4° C. for the purpose of storing reagents or media. The platform 100 may include other components not illustrated in FIG. 1, such as interior/exterior interfaces for manual intervention (e.g., sterile glove ports) or for moving objects in and out of the platform 100 (e.g., load locks). The platform 100 may be built in an isolator model, in which the interior is aseptically separated from the external environment, or in the form of a biosafety cabinet, in which the interior is accessible but protected via airflows designed to prevent external contamination from entering. In some implementations, the platform 100 may include more than one optical engine 104 and/or fluid management system 106.

Bioprocessing Systems with Pluggable Cassettes

The systems and methods disclosed herein include a series of systems for bioprocessing that are enabled by a pluggable, self-sealing (or normally-closed) fluidic cassette format. The cassettes include an automation-compatible mechanical carrier, internal fluidic portions, and fluidic connectors that are normally sealed to the external environment. The majority of the internal fluidic system may be filled with liquid such that orientation or motion of the cassette causes little internal liquid motion. There are a variety of advantages to the self-sealing pluggable cassette described herein. For example, such cassettes have the advantages of the fluidic chambers disclosed herein in terms of stability, control of conditions, superior image quality, and resistance to contamination, but also allow very flexible bioprocesses and equipment by enabling a variable number of chambers to be used in a process because fluidic handling may be done centrally. The cassettes minimize the amount of overhead fluid that is trapped in permanently connected tubing, and which must often be flushed from the system prior to use with cells. Furthermore, the cassettes enable equipment to be better shared between multiple fluidic chambers, and enable bioprocesses that use a series of fluidic chambers for a series of phases or operations, including chambers with different geometries, surface properties, or other functions. The cassettes also enable flexible and high-acceleration transport around equipment and systems, for example in systems with shared pieces of equipment. Lastly, the cassettes potentially enable a range of different functions on a cassette with a standardized pluggable interface.

Additionally, the present implementations include aseptic versions of the self-sealing pluggable cassettes as described herein, which have further advantages. For example, the aseptic cassettes may enable multiple batches, such as patient-specific batches, to be processed in the same environment (room, or system) without the possibility of cross-contamination. They also enable transport and handling of cassettes in non-sterile, low-grade cleanrooms, or even “controlled not classified” (CNC) facilities. This allows transport between different systems for the purpose of accomplishing different process phases, or for switchover in case of equipment downtime. Additionally, it allows transfers between automated systems and manual (isolated) steps where needed. The aseptic cassettes may also enable cassette-to-cassette coupling and/or materials transfers.

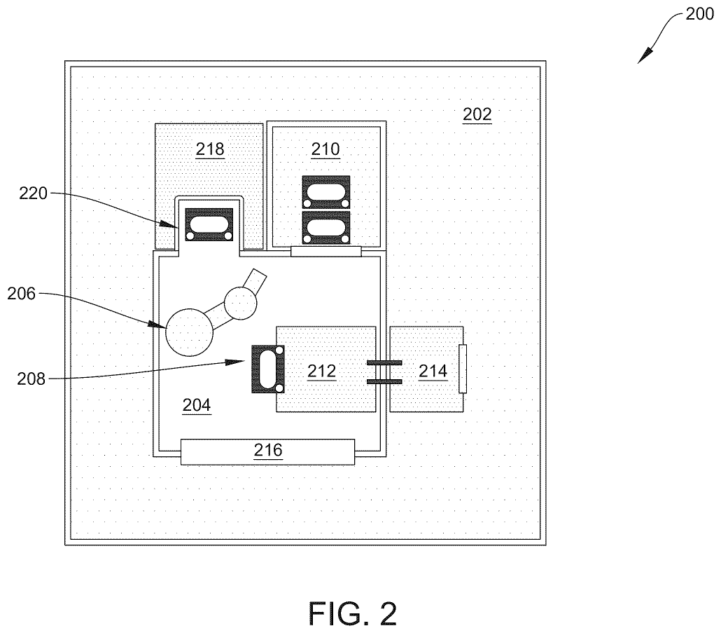

FIG. 2 is a diagram of an operating environment 200 (e.g., system 100) enabled by pluggable fluidic cassettes in accordance with various implementations. The environment 200 may be configured to support a single cell batch (for example, autologous patient batch) per environment. The present implementations detail the use of the environment 200 in a clinical process according to current good manufacturing practices (cGMP), in which a cell batch is processed in a dedicated ISO Class 7 cleanroom 202. Within this cleanroom 202, there is a workcell 204 with a clean environment with air handling to create an ISO Class 5 sub-environment (equivalent to a biological safety cabinet, or BSC) to serve as the core of the semi-automated system. Within the workcell 204, an automated transport system 206 is positioned to move cassettes around to different subsystems. In some implementations, the transport system 206 may be a multi-axis robot for maximum flexibility. The transport system 206 may, besides moving cassettes to different subsystems, also provide mechanical actuation functions such as “tapping” or “rotation” that have been described herein.

The workcell 204 also includes a plurality of fluidic cassettes 208, each cassette having a cell growth or processing chamber and at least two normally-closed pluggable ports (for example, as illustrated in FIG. 39A). The use of this consumable format allows robotic transport without sloshing of contents. In other words, the closed fluidic cassette format constrains media and cells between two surfaces and greatly reduces fluid flow, shear stress, and agitation during typical cassette manipulations, such as removal from an incubator by a fast-moving robotic arm. The cassette format also allows use of the operating environment 200, including automated incubator 210, without high humidity normally required when using open-top consumables. In this BSC-equivalent design, the incubator 210 may maintain a temperature of 37° C. or other desirable cell culture temperature, as well as CO₂ and/or O₂ concentrations. The incubator 210 may be maintained at low humidity to prevent growth of potential contaminating organisms. This is enabled by the sealed nature of the pluggable cassette, with most of its components largely impermeable to water vapor, while in some cases allowing gas exchange through a component such as a clear polymer window, as described herein.

The workcell 204 also includes a liquid handling system 212 that is configured to transact with the cassettes 208 to change cell media, perform washings, seed or harvest cells, etc. Interfacing and liquid transactions with the cassettes 208 may be performed with disposable pipette tips, as described herein. Connected to the liquid handling system 212 is, optionally, a media storage and supply system 214 configured to maintain media at 4° C., and warm and/or equilibrate media as described herein. This system 214 allows access by the user to exchange liquids as indicated by the door on its right face. The system 214 may also maintain a reservoir of phosphate-buffered saline (PBS) or other liquids for washing operations. Waste liquid may also be drained to a parallel storage container. The fluid supply system 214 may additionally maintain a supply of 70% isopropyl alcohol or similar cleaning agent if the overall system is configured to perform self-cleaning operations, for example of the workspace, or portions of the fluidic cassettes 208, on a regular basis. A sash or door 216 may be used to manually load or unload consumables and tubes with cells, small-volume liquids or reagents, with appropriate cleanliness precautions (when used in a clinical setting) such as would be used when using a BSC in a cGMP cleanroom.

The workcell 204 also includes an optical engine 218 configured to provide imaging functions, and in some implementations, laser scanning functions that selectively process (including kill) cells within the fluidic cassettes 208. An aspect of the present implementation is that the workcell 204 includes a clear box 220 that protrudes into the optical engine 218, that allows imaging and scanning using the optical engine 218 that is external to the interior of the workcell 204. This isolates the optical engine 218 from biological materials. The present implementations allow the partial automation of a range of bioprocesses, in which imaging, scanning, washing, media changes, harvest, etc. may be performed autonomously or via remote supervision. This drastically reduces the amount of hands-on labor, which involves extensive gowning and decontamination routines. It also allows one skilled biologist to review images and cell maps from multiple such units remotely, rather than entering the cleanroom. The lower staffing in the cleanroom in turn increases cleanliness and reduces the chance of contamination. In addition, the use of closed fluidic cassettes 208 with normally-closed pluggable ports drastically reduces the possibility of contamination, while also providing a more consistent operating environment, allowing maximum control of the process via imaging, mapping, software algorithms, expert viewing, and laser cell removal. It also enables precise, well-controlled fluidic transactions that have much more predictable shear forces, media and particle distributions, etc. than open-container liquid transactions.

For example, to accomplish a weekend of bioprocessing on eight cassettes carrying cell cultures, 32 disposable pipette tips may be placed into the enclosure prior to the weekend (four pipette tips per cassette). Remote monitoring may be performed via images from the optical engine 218, sensor readings from various workcell components, and/or cameras installed in the workcell 204. The workcell 204 and the surrounding environment (cleanroom) may be sterilized and cleaned extensively between cell batches. The environment 200 and its constituent systems may run using either non-aseptic or aseptic normally-closed fluidic connectors on the cassettes 208. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

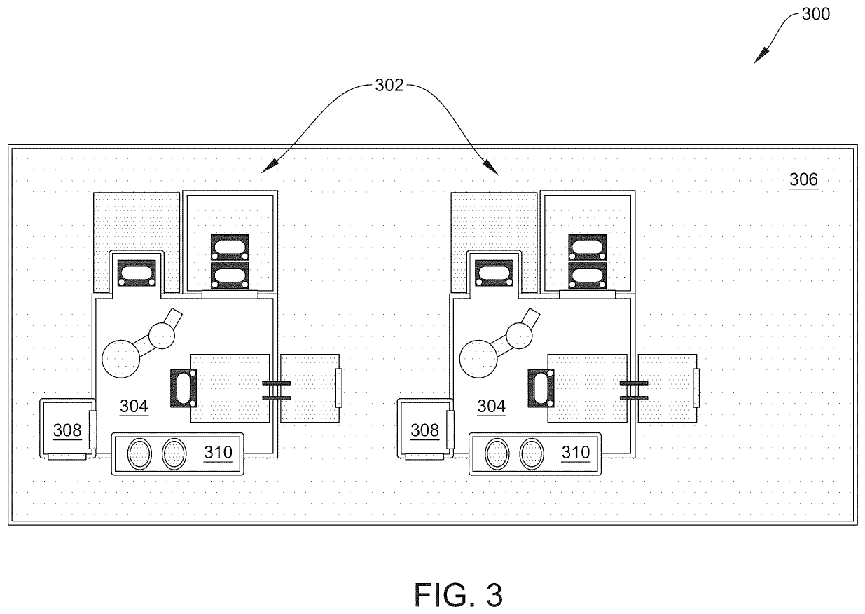

FIG. 3 is a diagram illustrating another operating environment 300 for performing batched bioprocesses (e.g., autologous bioprocesses) in accordance with various implementations. The environment 300 includes a plurality of semi-automated isolator workcells 302 that operate using normally-closed fluidic cassettes (e.g., the cassettes described with reference to FIG. 39A). The workcells 302 may be similar to the workcells described with reference to FIG. 2. In present implementations, the primary workspace where cassettes are handled and transported between workspaces 304 is isolated from the surrounding room 306, with access only through a load lock 308, which includes features for sterilization. For example, each load lock 308 may include a gas-based sterilization system. Supplies are bagged and placed into the load lock 308, a gas cycle sterilizes the containing bag, and after gas flushing, the supplies may be transferred to the isolated workspaces 304 and taken out of the bag, through use of the sterile glove ports 310. The glove ports 310 allow certain periodic operations (e.g., bagging, unbagging, opening/closing of vials, placement of consumables, etc.) to be performed manually to increase flexibility and keep the development cost of contained automation and robotics low. However, the majority of cell culture and processing operations may be run autonomously and/or under remote control.

An aspect of the present implementation is that because it operates as an isolator, the room 306 that constitutes the operating environment may be a lower-grade cleanroom, such as an ISO Class 8 cleanroom, or an ISO Class 9 cleanroom, or even a CNC environment. Importantly, multiple isolated workcells 302 of this design may be operated in the same environment 300, so that operators may service multiple workcells 302 and therefore multiple cell batches with a single controlled entry into the environment 300. This may also reduce the overall footprint of the cell processing facility. In some examples, workcells 302 may be operating the same bioprocess (for example, patient iPSC reprogramming) in parallel. In other examples, the workcells 302 may perform different portions of a process in a pipelined manner (for example, iPSC reprogramming, iPSC expansion, and iPSC differentiation into a target cell type). The internal portions of the workcell 302 may be sterilized between patient batches. In some implementations, all workcells 302 and the environment 300 may be sterilized and decontaminated simultaneously.

In some implementations, the isolator workcells 302 are designed to enable closed sterilization, allowing them to be sterilized while other workcells in the environment 300 continue to run bioprocesses. Sterilization may be performed by manual wiping of surfaces with liquids, and/or by gas-based sterilization such as a vaporized hydrogen peroxide (VHP) cycle. In some implementations, some components of the automated systems inside the workcells 302 may be replaced entirely with new components, or components that have been sterilized outside of the environment 300. The environment 300 and its constituent systems may run using either non-aseptic or aseptic normally-closed fluidic connectors on the cassettes. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

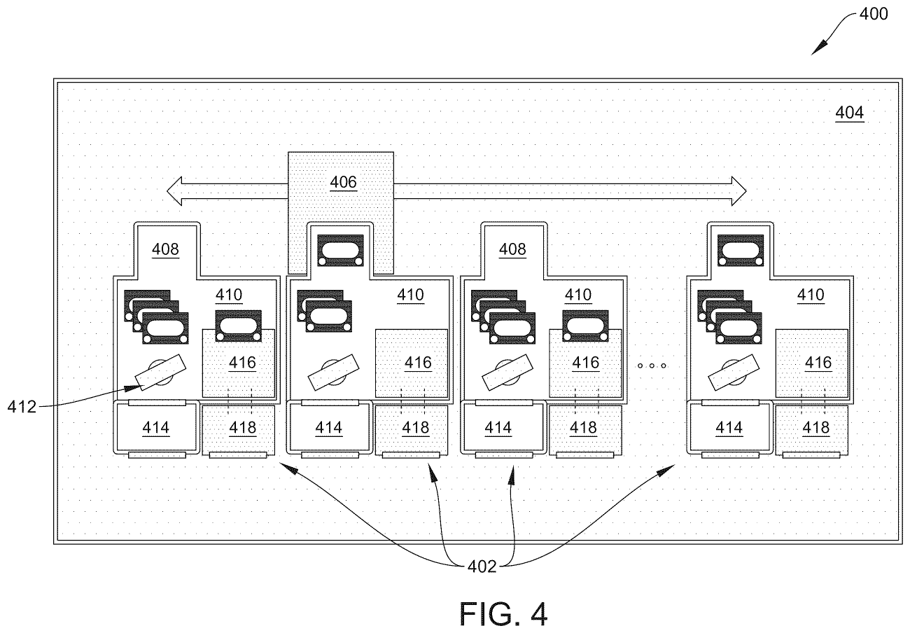

FIG. 4 is a diagram illustrating another operating environment 400 for pluggable cassette-based systems in accordance with various implementations. The environment 400 includes a plurality of single-batch processing workcells 402 used together in a single room 404, which may be a cleanroom, a low-grade cleanroom, or a CNC space. In the example shown in FIG. 4, a single optical engine 406 is shared among multiple workcells 402 to reduce overall costs. The optical engine 406 may be automatically translated into position based on an overall schedule for each workcell cluster. Different configurations may have a different number of workcells 402 per optical engine 406, including configurations in which each workcell has a dedicated optical engine. The optical engine 406 operates around an optical observation/treatment box 408 which is part of the isolated environment of each workcell 402. Multiple clusters or workcells sharing optical engines may share the same environment.

An isolated workspace 410 of each workcell 402 may be maintained at cell temperature and at appropriate gas concentrations, such that cassettes may be incubated in the central space rather than in a dedicated incubator. A transport subsystem 412 in each workcell 402 may be configured to move cassettes around the interior of the workspace 410, and load/unload items from load locks 414. Items may be pre-palletized such that automated loading, use, and unloading is possible. The transport subsystems 412 may be integrated with a liquid management system 416, such that a single set of actuators may be used for the entire lifecycle of a cassette in the workcells 402.

The liquid management systems 416 may be connected to liquid/reagent/waste storage subsystems 418 for each workcell 402. This may be done via tubing, or via a pallet-based setup and load lock with sterilization as described herein. The workcells 402 in this implementation may include capping/decapping and other tube handling automation to automate transfers of small amounts of reagents, cells, or liquid samples for analysis of ongoing bioprocesses. The environment 400 and its constituent systems may run using either non-aseptic or aseptic normally-closed fluidic connectors on the cassettes. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

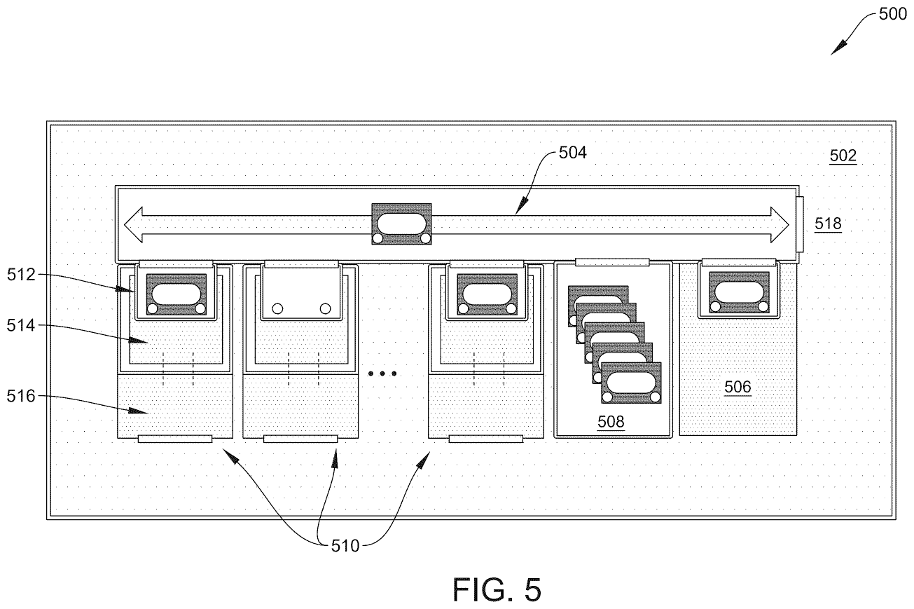

FIG. 5 is a diagram illustrating another operating environment 500 utilizing shared equipment by multiple cell batches in accordance with various implementations. The implementation shown in FIG. 5 is dependent on reusable aseptic pluggable connectors on cassettes for the purpose of preventing cross-contamination between batches when using shared equipment. The operating environment 500 includes a room 502 that may be a low-grade cleanroom or CNC facility, and a transport plane 504 configured to move cassettes, around which a series of process modules are arranged. Multiple systems configured around multiple backplanes may share the same environment. The modules are designed in a manner that allows a system to be flexibly configured, and modules may be exchanged at will, even during operation, both for maximizing uptime and reliability, and for bioprocess flexibility. Modules may include but not be limited to: one or more optical engines 506 which provide imaging and/or laser scanning functionalities and/or other cell removal tool modules that enable cell removal within sealed fluidic cassettes; incubator modules 508 that incubate multiple cassettes, including in some cases cassettes carrying different cell batches (for example, batches belonging to different patients in autologous bioprocesses); and fluid management modules 510 which may be batch- or patient-specific fluid management modules.

Each fluid management module 510 may include a cassette handling area 512 which includes an aseptic connector system that attaches to the cassette as described herein and a fluidic handling system 514 which manages fluidic and gas transactions with the cassette, and manages other functions such as mixing, washing, cell seeding, cell harvesting, etc. The fluidic handling system 514 is attached to a fluidic storage subsystem 516 which stores media, reagents, washing liquids, cell samples, etc. as described herein. The fluidic storage system 516 is accessible externally to facilitate changes in liquids and loading/unloading of reagents and consumables, where applicable. This may be accomplished by bulk bags/containers attached via tubing, and/or individual containers that are presented to the fluid handler. Cassettes may be loaded or unloaded into each module via door 518 at the end of the cassette transport plane 504. The transport, incubation, and other portions of the system will generally be kept clean using filtered airflow, for example at ISO Class 7 or even ISO Class 5 levels, but this is only an extra precaution, because the aseptic nature of the cassettes allows them to be handled in CNC spaces. This includes transfers from one system to another, in the case that a bioprocess is accomplished on multiple systems, or in cases in which cassettes are switched over from one system to another for maintenance, cleaning, or repair of the system. The modular nature of the system, however, allows individual modules to be maintained in place, or detached and maintained, without interrupting the operation of the system.

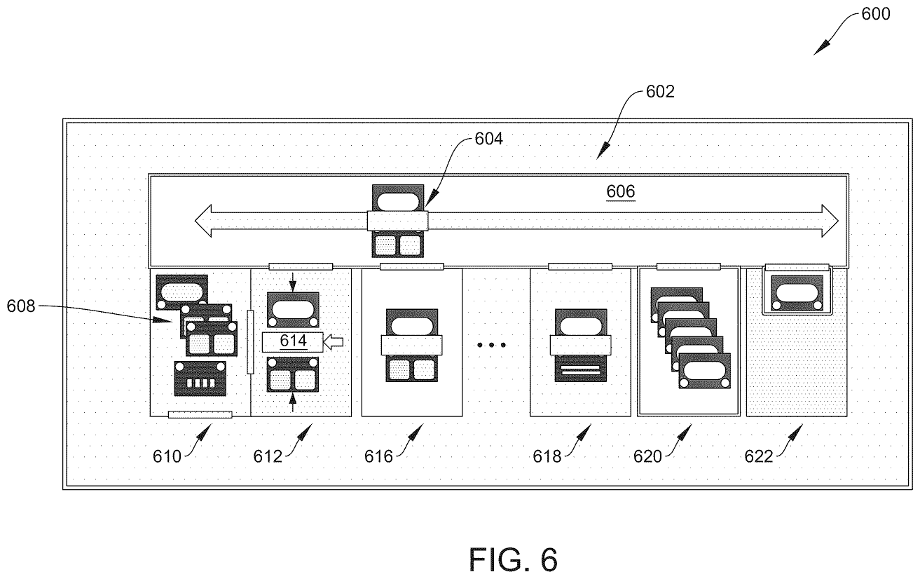

FIG. 6 is a diagram illustrating another operating environment 600 supporting an aseptic normally-closed pluggable cassette-based system 602 in accordance with various implementations. Multiple such systems may share the same environment. The system 602 operates based on an aseptic coupling concept in which cassettes are coupled together and transported to various process modules in coupled configuration 604. The coupled cassette configuration may include a fluidic cell culture cassette (top cassette) and a media/waste cassette (bottom cassette) attached to an intervening coupler. In this configuration, the coupled cassettes 604 may be aseptically sealed to one another, and fluids, gases, cells, etc. may be exchanged for various operations. Cassettes, either individually or in aseptically-coupled form, are transported via backplane 606 that connects multiple functional modules, to provide a highly configurable and reliable system architecture.

Cassettes 608 are loaded via an access port on a cassette management/storage system 610, which may include multiple temperature zones, etc. Multiple types of cassettes may be loaded, as described herein, including cell growth cassettes and media and waste cassettes, which may be single-use (i.e., perform a single media change on a single growth cassette), multi-use cassette-dedicated (i.e., perform multiple media changes on a single cassette over a period of time), and/or multi-use batch-dedicated (i.e., perform multiple media changes on multiple cassettes containing the same cell batch). The cassettes 608 may also include function-specific cassettes, which can provide a range of functions including but not limited to cell sorting, filtration, intracellular delivery, spheroid or droplet formation, etc.

One or more aseptic coupling modules 612 communicates with the cassette storage system 610 and transport backplane 606, and provides aseptic coupling and uncoupling functions between pairs of cassettes. This may include the mechanical connection of the cassettes to a coupler 614, sterilization routines (via gas, liquid, heat, plasma, UV, etc. as described herein), and sterile liquid connections between the two cassettes. In some implementations, 3+ cassette coupling formats are contemplated, for example in which multiple cassettes in a single batch are connected to one media or cell source cassette. In some implementations, the coupling modules 612 may include storage for cassettes that are in the process of being sterilized, in which the sterilization action requires residence of a chemical sterilizer (or in other implementations may be a separate module in the system 602).

Once coupled with the sterile liquid/gas connections established, the ensemble may be transported to one or more functional modules 616, which may for example represent a media exchange module, in which media is warmed and equilibrated and then pushed into the cell growth cassette, with the waste collected. Other modules 618 may handle other operations, which may include different pumping, valve actuation, electrical or optical connections to specialized modules, imaging, laser systems, etc. to accomplish a wide range of operations. In this manner, a large range of complex bioprocesses may be accomplished using a common system architecture and aseptic connection system, by adding special-purpose cassettes and functional modules. The system shown in FIG. 6 enables many different bioprocesses to be supported by adjusting the configuration of modules and cassettes, and running different processing routines. Other modules including incubators 620 and optical engines 622, as described herein, may also be connected to the system 602. Specialized incubators that utilize coupled cassette pairs for continuous perfusion media exchange (and temperature control of the two cassettes) may be used. Similarly, the optical engine design may include accommodation for coupled cassettes, in which imaging observation and/or laser processing may be combined with fluidic operations such as washing.

### Active Cell Expansion Management

The present implementations cover methods for pre-patterning cells prior to passaging (i.e., transferring from one cell growth container to another), including through the use of cell removal tools (CRTs) as described herein, including but not limited to laser-based tools. The present implementations additionally cover methods for managing colonies within cell culture containers, including but not limited to fluidic growth chambers and cassettes. These methods include methods for expanding selected colonies in an efficient manner that is compatible with closed fluidic cassettes, including methods for controlled mini-expansion of select colonies to ensure consistent and healthy cell densities while growing the total number of cells. Additionally, methods are disclosed for optimally efficient cell expansion (e.g., maximizing expansion rate per passage step) and optimal use of growth surface, and methods for spatial patterning prior to differentiation steps.

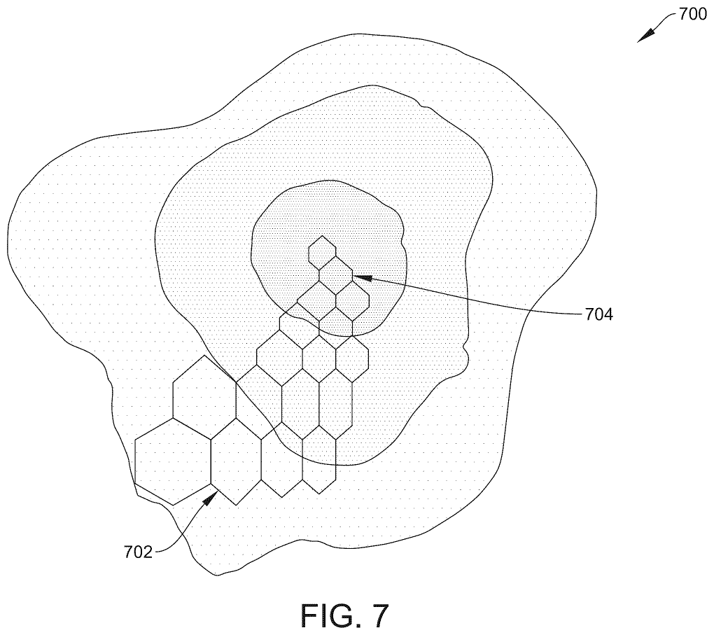

FIG. 7 is a diagram illustrating a method of pre-patterning cell colonies in accordance with various implementations. The pre-patterning may be performed by cutting all or a portion of a cell colony 700 into patches, each containing a roughly equal number of cells. The cell colony 700 may contain regions of varying cell density. The goal of the pre-patterning method is to provide cell clusters of roughly equal cell count for downstream processes, such as seeding onto a new surface or culturing in suspension. The cell colony 700 may be cut, by a CRT in a grid pattern, into larger islands 702 in low-density regions and smaller islands 704 in high-density regions, resulting in clusters of roughly consistent cell count. A variety of patterns may be used to provide a combination of cell health (e.g., leaving cells with the appropriate number of neighbors, such as more circular or hexagonal patches if a large number of neighbors is desired), minimizing “kerf loss” (i.e., the width of material removed by the CRT) (for example, kerfs of ≤100 microns, or ≤75 microns, or ≤50 microns or even ≤25 microns), and maximizing the ease of harvesting the resulting islands using chemical and/or mechanical means.

In some implementations, the CRT cut lines may advantageously leverage mechanical forces of cell-cell interactions or cell growth patterns. For example, if cells are elongated and directional, islands that are longer in the longer axis of the cells may be cut (analogous to cutting along the grain of a material). The CRT used to pattern the cell colony may include, but not be limited to, a laser system that uses a pulsed laser to interact with a semi-transparent film on which the cell growth occurs. In some implementations, a wait time is imposed between when the cell sheet is patterned and when the cell clusters are harvested, to allow cells to become more dense within the patterned islands. Such densification, which may improve intact removal of the islands, may be promoted by CRT removal of the extracellular matrix (ECM) in the cut lines. In some implementations, the pattern may be applied multiple times to kill/remove cells and/or ECM while keeping the individual cut actions as stress-free as possible for the surrounding remaining cells. In some implementations, dense islands of cells cultivated this way may be used for in-process testing (e.g., having the cells undergo differentiation and then harvesting them for quality control).

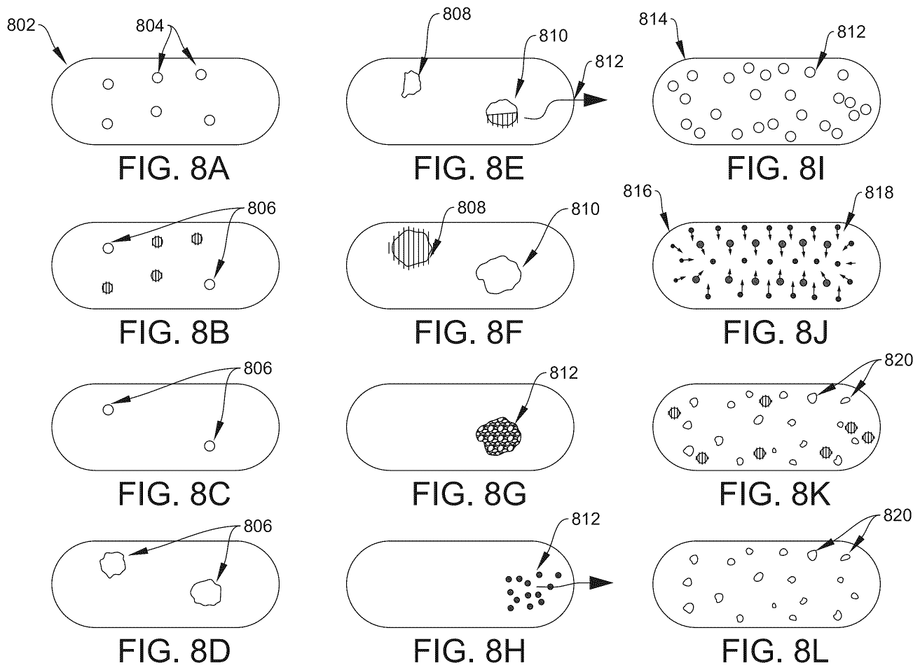

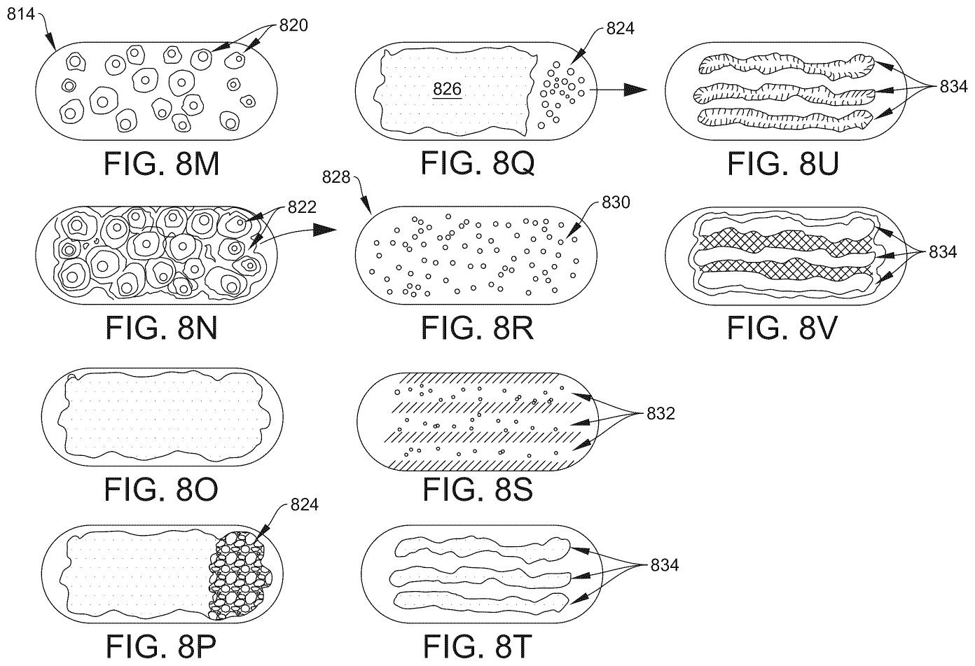

FIGS. 8A-V are diagrams illustrating an example series of cell culture process operations in accordance with various implementations. FIG. 8A depicts a cell culture container 802, which may be a fluidic chamber (e.g., a fluidic closed cassette), containing a plurality of cell colonies 804 growing on a cell culture surface of the container 802. The cell colonies 804 may be, for example, clonal induced pluripotent stem cell (iPSC) colonies that have been managed on the cell culture surface for an extended period after reprogramming and/or gene editing to stabilize the cells and clear any reprogramming vector. This maintenance may include feature tracking that is used to predict clonal quality and rank the clones, as described herein. In the case of reprogramming, these colonies may have grown directly out of the reprogrammed material, either in the same cell culture container or previously passaged from another “early emergence” cell culture container. In some implementations, multiple separated cell colonies 804 may be density-managed by CRT at local colony densities that allow in situ cell density management without colony-colony collisions, for example at colony densities of ≥0.5/cm², ≥1/cm², ≥2/cm², or ≥5/cm², allowing, in contrast to conventional clonal cell culture techniques, more than one clone per container to be managed and observed, for example ≥2 clonal colonies per container, ≥3 clonal colonies per container, ≥5 clonal colonies per container, and even ≥10 clonal colonies per container.

FIG. 8B depicts removal by a CRT of most of the cell colonies shown in FIG. 8A, except for the top two clonal cell colonies 806. The cell colonies 804 may be ranked based on predictions of colony quality, either by a machine learning model or by human inspection, or a combination of automated and manual evaluation. In the example shown in FIG. 8B, only two cell colonies 806 are retained, but in general a certain percentage of colonies may be retained after CRT removal. For example, less than 50% of cell colonies may be retained, or less than 25%, or less than 10%. FIG. 8C shows the retained cell colonies 806 after cell debris (i.e., the other cell colonies removed from the cell culture surface) are washed away. FIG. 8D shows the retained cell colonies 806 after a period of time when they are allowed to expand in area and cell count. The growth characteristics of the clonal colonies 806 may be tracked for later use, for example using an optical engine and a platform manager of a cell manufacturing platform. Methods of tracking cell growth and dynamics such as those described herein may be used to track behavior of each colony.

FIG. 8E depicts the cell culture container 802 after a portion of a second-ranked cell colony 808 has been removed to maintain healthy cell density (and debris has been cleared from the cell culture container) and a portion of a top-ranked colony 810 is removed by the CRT and the resulting cell debris 812 is retrieved from the cell culture container 802 for analysis. The analysis may include but is not limited to genomic integrity testing, DNA sequencing, RNA sequencing, PCR analysis, proteomic analysis, etc. In some implementations, the CRT may be used to selectively remove ECM to create boundaries that limit the growth of cells into areas adjacent to colonies. In one such implementation, a “moat” of ECM-free area may be formed around a cell colony, and as the colony becomes dense within the confined area, cell-cell adhesion outweighs cell-ECM adhesion, and the colony eventually floats off the surface. The resulting live cells may be removed from the cell culture container for downstream culture or analysis, including but not limited to the methods above. In some cases, the cells may form a 3D spheroid or embryoid body that can be differentiated. In other implementations, an ECM “moat” may be formed proximate to a cell colony or region to check for the ability of cells to propagate into that moat, either under standard media conditions, or under some modulation with signaling factors. The propensity of cells to migrate/propagate into the ECM-free or ECM-poor region may be correlated with pluripotency, or ability to differentiate into specific lineages, and may then be used to rank, score, or select cell colonies. The purpose of the analysis may be to confirm the quality of the top-ranked colony 810, or to indicate an issue with it and proceed with the second-ranked colony 808. FIG. 8F depicts removal of the second-ranked cell colony 808 using a CRT, leaving the top-ranked cell colony 810 (the quality of which may be confirmed by the harvested debris assay). The debris from the second-ranked cell colony 808 may be washed away from the cell culture container 802.

FIG. 8G depicts the selected top-ranked cell colony cut into clusters 812. The clusters 812 may be cut to each contain a target number of live cells, as described with reference to FIG. 7. The target cell count per cluster may be previously determined by experiments harvesting these clusters from the cell culture container 802 and seeding the clusters onto a new cell culture surface, and analyzing the clusters in terms of survival rate and/or target cluster size in the downstream cell culture container. FIG. 8H depicts harvesting of the clusters 812 out of the cell culture container 802. The harvest may be performed using chemical and/or mechanical means. In some implementations, the pre-cutting of the clusters 812 using the CRT may facilitate the harvest, or improve the health of the harvested clusters.

FIG. 8I depicts a new cell culture container 814 into which the clusters 812 have been seeded. In some implementations, the same cell culture container may be used, with cells or cell clusters being redistributed over the entire growth surface of the container. In some implementations, the clusters may include individual cells. In alternate implementations, the clusters may include multiple attached cells. The clusters 812 attach to the cell culture surface of the cell culture container 814 and proliferate. Through imaging and computer vision models (including but not limited to deep learning models), the position, area, and area growth rate and directionality (derived from time-series images), if any, may be determined for each cluster or region of clusters. FIG. 8J depicts a map 816 of the cell culture container 814 based on previous cell expansion observations. The map 816 shows the average cell expansion rate as well as directionality, as represented by the point sizes and arrows 818, respectively. Spatial variations in these characteristics may result from a range of effects, including non-uniformities in the ECM coating of the surface, non-uniform nutrient distribution or waste removal in cell media, non-uniform dissolved gas concentrations, non-uniform effects of fluid flow profiles, spatially-varying mechanical or thermal variations, etc. Local cell/colony behaviors may have been tracked via methods described herein for back-tracing and characterizing cell dynamics. In other implementations, optical flow-style image processing algorithms may be used on live cell or cell density maps to estimate net proliferation and/or net motion of cells by region in the cell culture container.

FIG. 8K depicts selective removal of a subset of clusters based on a map computed to achieve the highest uniformity of cell confluence at the end of cell culture expansion, leaving retained clusters 820. The map may be computed from information on the overall clonal behavior observed in prior steps of the process (e.g., area proliferation rate, cell proliferation rate, cell division rate as a function of cell density, etc.), the cluster-specific features observed after seeding and initial growth, optical flow measurements, and/or the spatial map 818 of past cell expansion characteristics. The map may be generated from a heuristic model, from a series of simulations, or from a reinforcement learning model that has been trained on prior runs and/or simulated runs. In some implementations, clusters may be partially removed to reduce cell count, and therefore tune when a cluster is expected to achieve a target size. In other implementations, the ECM may be selectively removed from regions around a cluster to direct/shape the growth of the cluster. The objective of all the actions performed by the CRT is to set up the earliest stage of expansion to achieve the most uniform possible growth over a large part of the cell culture container 814.

FIG. 8L depicts the cell culture after the selective cluster removal shown with reference to FIG. 8K, leaving the retained clusters 820 to proliferate. The retained clusters 820 may have been modified by the CRT and/or the surrounding regions may be modified by the CRT, for example having ECM partially or completely removed. FIG. 8M depicts the cell culture after proliferation, showing enlarged retained clusters 820. FIG. 8N depicts the cell culture as it approaches maximum confluence. As the culture approaches confluence, some regions 822 (often corresponding to the original cluster centers) may become over-dense. In such a case, the CRT may be used to remove the over-dense regions 822 so that these regions do not spontaneously detach or differentiate. An arrow indicates that during such a de-densifying procedure, the resulting detached cells or cell debris may be collected for external analysis, as described herein. Additionally, the CRT may be used to remove regions showing abnormal or undesirable characteristics or dynamics, whether identified by cell or colony morphology, or cell/colony dynamics as tracked by tools described herein or similar.

FIG. 8O depicts the cell culture after further expansion which, due to cell culture management using the CRT from the start of the expansion, may achieve a high confluence without the formation of over-dense regions. Cells may be maintained at healthy densities, for example ≤10,000 cells/mm², ≤5000 cells/mm², ≤5000 cells/mm², or ≤4000 cells/mm² locally for pluripotent stem cells, and with high confluence across the overall growth area, for example ≥70% confluence, ≥80% confluence, ≥90% confluence or ≥95% confluence. This ensures maximum area efficiency while maintaining uniform, healthy cell environs.

FIG. 8P depicts the expanded cell culture after it has been cut into islands/clusters 824 with a CRT. FIG. 8P shows a scheme in which the cell culture sheet is scored in a manner that minimizes the stress and inconsistency in harvesting according to local features. The local features may include, for example, cell density, how long cells have been resident on the ECM at a particular location, and/or proximity to the edge of a contiguous cell sheet. All of these factors may impact the level of attachment to the cell culture container surface, and the requirements for harvesting them successfully. In this manner, the conditions for optimal harvesting are made more uniform, and less stressful (e.g., lower concentrations and/or shorter durations in dissociation agents, less mechanical action or fluidic shear stress required, etc.). In addition, the harvesting pattern shown in FIG. 8P is a partial harvest of the cell culture, defined at least in part by the CRT pre-cutting of the cell sheet (in some implementations, it may also involve spatially-varying application of reagents and/or fluidic/fluidic-air shear stresses). This partial harvest may be performed, for example, to achieve a specific concentration of cells/cell clumps per volume of liquid. This may be advantageous for harvesting in fluidic components (tubing, pipettes, valves, etc.) without clogging. This may also be advantageous for the purpose of achieving a target cell concentration for downstream processes, such as re-seeding. Harvesting may be achieved using liquid flows/shear stresses, liquid-air interface flows, mechanical vibrations, dissociation agents, and/or CRT-based actions.

FIG. 8Q depicts the now detached cell clusters 824 being harvested out of the cell culture container 814, while the remainder of the cell culture 826 remains adhered in the cell culture container 814. FIG. 8R depicts a new cell culture container 828 into which the harvested cell clusters 824 from cell culture container 814 are seeded. The seeded cells 830 may be seeded as single cells, for example, in preparation for a differentiation protocol to follow. FIG. 8S depicts a patterning applied via CRT to the cell culture container 828 to, for example, prepare for a high-yield differentiation protocol resulting from better spatial control or to control the cell proliferation rate that is optimal for differentiation. Cells are allowed to remain in several linear regions 832, while the cells that seeded in the areas between these are removed by the CRT. In addition, the non-cell areas may be fully or partially treated with the CRT to remove ECM. The patterning of the cells and ECM may be driven by previously observed growth characteristics of the source cells that have been seeded into the cell culture container 828, on a time series observation of the initial growth of these cells, on known growth patterns in the cell culture container 828, and/or on differentiation characteristics predicted (or previously measured) for the particular cell line, clone, or patient.

FIG. 8T depicts the seeded cells after expansion into cell regions 834, arranged in a manner to support good source cell health (for example, a sufficiently wide band of pluripotent cells to support good pluripotency), but also as to be optimally set up for differentiation, which in the example shown in FIG. 8T occurs in a spatially-dependent manner. The cell culture may be monitored via imaging by an optical engine of a cell manufacturing platform. Based on maps of the cells computed from these images, and potentially previous cell behavior and/or assay data, the differentiation process may be initiated, for example with the change of the cell media and its constituents. FIG. 8U depicts the cell regions 834 undergoing initial differentiation around the edges of the patterned regions. FIG. 8V depicts the next stage of differentiation, in which the cell regions 834 between the initially formed islands have differentiated into the target cell type. Thus, FIGS. 8A-V depict an example of utilizing CRT patterning, including patterning determined using a predictive model, to enhance the efficiency of cell differentiation processes.

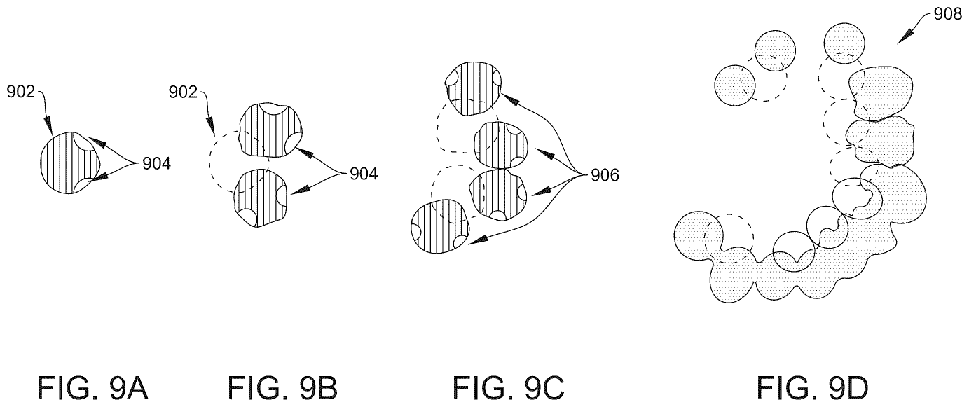

FIGS. 9A-D are diagrams illustrating an example method of an in-place expansion sequence for a cell colony in accordance with various implementations. An initial cell colony 902 growing on a cell culture surface is reduced into two sub-colonies 904 using a CRT. This allows a healthy low cell density to be maintained, and if ECM is destroyed/removed by the CRT, cells are translated towards regions with fresh ECM. FIG. 9B depicts proliferated colonies 906 that grew out of the sub-colonies 904, as well as an outline of the initial cell colony 902. The proliferated colonies 906 may be allowed to proliferate until the total area and number of cells have approximately doubled from the initial colony 902. Additional CRT operations may be applied to each colony 906 similar to the CRT operation performed in FIG. 9A, resulting in two smaller sub-colonies for each colony 906 (or four sub-colonies in total).

FIG. 9C depicts another iteration of proliferation and CRT removal of the four sub-colonies generated in FIG. 9B. This yields four colonies 906 that are each approximately the size of the initial colony 902, each with healthy cell densities. CRT operations may be applied again to the colonies 906, leaving two sub-colonies per colony. FIG. 9D depicts the result of another iteration of proliferation, in which the eight sub-colonies from FIG. 9C have formed a ring structure 908. Thus, by repeated iterations of expansion and CRT removal, the initial colony 902 has been proliferated significantly while (a) maintaining healthy cell density throughout; (b) not involving cell passaging or other disruptive steps that force re-plating of cells; and (c) moving the cells in a manner that allows one-time use of ECM surface for cell growth (i.e., the sub-colonies are translated relative to their parent colonies and proliferate into new areas of the cell culture surface where CRT operations have not damaged the ECM).

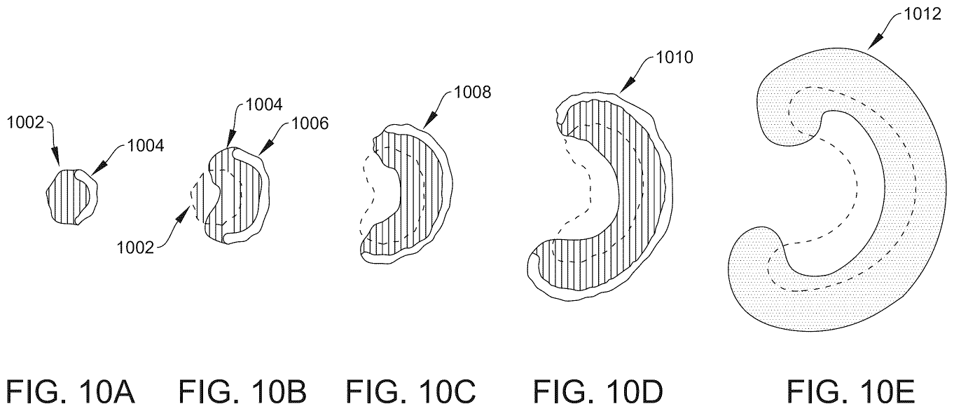

FIGS. 10A-E are diagrams illustrating another example method of an in-place expansion sequence for a cell colony in accordance with various implementations. FIG. 10A depicts an initial cell colony 1002 growing on a cell culture surface is reduced in size by a CRT operation, leaving a crescent-shaped sub-colony 1004. The sub-colony 1004 may be crescent-shaped to encourage outward growth from the cell removal region, particularly if the ECM is removed by the CRT during cell removal. FIG. 10B depicts outlines of the initial colony 1002, as well as the sub-colony 1004 after expansion. Another CRT operation may be applied to the expanded sub-colony 1004, leaving another crescent-shaped sub-colony 1006 that is larger than sub-colony 1004. FIGS. 10C-D show additional iterations of proliferation and CRT removal operations, resulting in increasingly larger sub-colonies 1008 and 1010, respectively. Via the in situ colony expansion methods described herein, a colony may be expanded on a single surface and inside of a single container, while maintaining healthy cell densities throughout. For example, a colony may be expanded from ≤10 cells, from ≤100 cells, from ≤1,000 cells, or ≤10,000 cells; it may be expanded in situ to ≥1,000 cells, to ≥ 10,000 cells, to ≥100,000 cells, or even ≥1,000,000 cells by these methods. During a cell culture process it may be maintained at densities that are healthy for the specific cell type, for example ≤10,000 cells/mm², ≤7,000 cells/mm², ≤5,000 cells/mm², or ≤4,000 cells/mm² to promote healthy proliferation and/or pluripotency.

FIG. 10E depicts a final iteration of proliferation and CRT removal that results in a crescent-shaped colony 1012. Thus, the initial colony 1002 has been proliferated significantly while (a) maintaining healthy cell density throughout; (b) not involving cell passaging or other disruptive steps that force re-plating of cells; and (c) moving the cells in a manner that allows one-time use of ECM surface for cell growth (i.e., the sub-colonies are translated relative to their parent colonies and proliferate into new areas of the cell culture surface where CRT operations have not damaged the ECM).

While FIGS. 9A-D and 10A-E show example CRT removal patterns and retained sub-colony shapes (e.g., circular or crescent-shaped), in general any sub-colony shape and removal patterns are contemplated herein. For example, in some implementations the ECM may not be damaged by CRT operations or the ECM may be refreshed after CRT operations. This allows additional proliferation and removal iteration strategies. In one example, the CRT operation may remove the dense center of a circular cell colony, leaving a ring-shaped sub-colony that is allowed to proliferate both outwardly onto fresh ECM and inwardly towards the center onto the undamaged or refreshed ECM.

During in-place colony expansion, the tracking and dynamics measurement methods described herein may be used to collect features of the colony, for example, cell motility, colony and/or cell morphology, cell proliferation rate, density, spontaneous differentiation rate, etc., that may be used to characterize or rank colonies. In some implementations, this process may be combined with modulation of conditions to further elucidate colony state, phenotype, quality, viability, etc. Furthermore, the measured characteristics may be adjusted for location within the cell culture container as described herein.

### Systems and Methods for IPSC Reprogramming in Pluggable Cassettes

Consistent production of induced pluripotent stem cells (iPSCs) for regenerative medicine, drug discovery, and/or developmental biology modeling is a significant challenge. A “one size fits all” process with fixed numbers of input cells, fixed numbers of clones produced, fixed timing for clone picking, fixed duration and timing for expansion, and fixed number of cells produced per clone is an extremely fragile process that often results in low yields for a variety of reasons. These reasons include a high degree of patient-to-patient variability in source material quality, reprogramming efficiency, iPSC clone stability, survival and proliferation, and quality as measured by a range of assays, and ultimately differentiation capacity. In addition, the inherent (but also patient-to-patient and clone-to-clone variability) sensitivity and instability of iPSCs adds additional complexity. One result is that manually operated iPSC reprogramming processes are very complex, error-prone, expensive, and good manufacturing practice (GMP) operator staff are extremely difficult to recruit and retain. Approaches to autonomous iPSC reprogramming processes have been described, for example, in PCT Application No. US2023/030293, entitled “Systems and Methods for Cell Manufacturing” and filed Aug. 15, 2023, which is incorporated by reference in its entirety. The autonomous processes described therein enable some degree of adjustment to variability in iPSC clone behavior.

Another challenging aspect of iPSC reprogramming and/or gene editing for clinical applications (i.e., in a GMP environment and workflow) is the requirement to keep patient samples and/or clones isolated from one another, to prevent cross-contamination. Systems that enable closed cell processing and iPSC manufacturing can remedy this issue, for example, as described in U.S. Pat. No. 11,913,029, entitled “Platforms and Systems for Automated Cell Culture,” which is incorporated by reference in its entirety.

A problem remains, however, that currently iPSC reprogramming and/or gene editing processes mapped onto closed cell processing system designs do not provide a high degree of flexibility to account for all the sources of patient-to-patient and clone-to-clone variability described herein. An iPSC reprogramming/editing approach that combines autonomy, closed processes, and a higher degree of flexibility to respond to such variability is needed.

The present implementations disclosed herein include an iPSC reprogramming process, which may also include gene editing, that is performed using systems and methods for operating bioprocesses in fluidic cassette formats, including in cassettes with normally closed fluidic ports and aseptic ports. The use of such pluggable cassettes and appropriate corresponding equipment for processing them enables a flexible workflow that can be tailored to patient-to-patient, clone-to-clone, or application-to-application differences in the iPSC reprogramming and/or gene editing process.

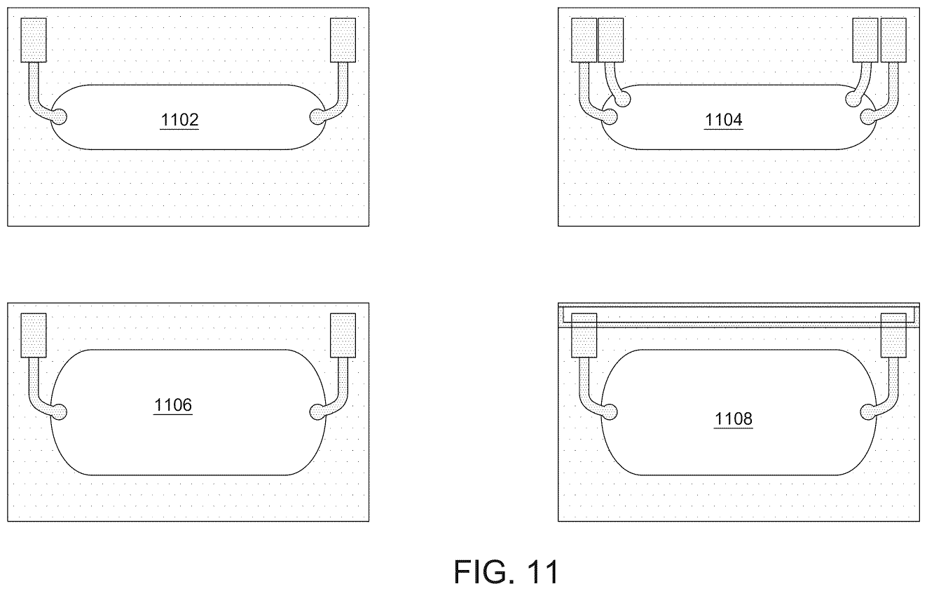

FIG. 11 is a diagram illustrating several normally closed pluggable fluidic cassettes for use in iPSC reprogramming processes in accordance with various implementations. For example, cassette 1102 has a smaller cell growth/culture chamber suitable for a smaller number of cells or colonies, and two normally closed (or self-sealing) fluidic ports that may be used to seed cells, exchange media and/or add reagents, do washing operations, and harvest cells. In another example, cassette 1104 has a two-level liquid chamber or channel in which the channels are separated by a semipermeable membrane or filter. The semipermeable membrane may be made of clear material such that imaging observations may be made in both the top and bottom fluidic chambers. These chambers are connected, respectively, to the inside and outside set of fluidic connectors. The cassette 1104 may be used to exchange media without disturbing or dislodging suspension or semi-adherent cells. In other implementations, the cassette 1104 may be used to concentrate cell material and/or to exchange the liquid containing cells without losing cell material, or deliver materials capable of diffusing across the membrane that are important for cell growth, expansion, and/or reprogramming. In another example, cassette 1106 is a cassette with larger surface area, for use in expanding cells. In another example, cassette 1108 is a large-area cassette that has been fitted with an aseptic connector such as those previously described herein, in which the normally closed pluggable connectors are protected by an external shield which is opened only in a controlled environment, typically after sterilizing the outside shield and any other components exposed to the environment. Such a shield may include, in some implementations, screw-on caps that may be sterilized prior to opening.

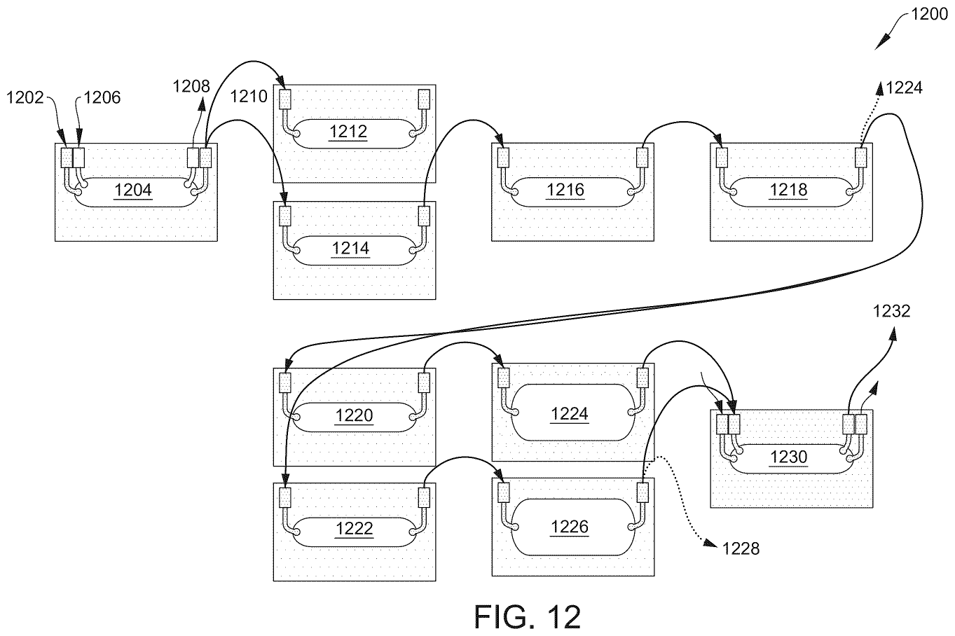

FIG. 12 is a diagram illustrating an example workflow 1200 using pluggable cassettes in iPSC reprogramming in accordance with various implementations. The cassettes may be aseptic connector type cassettes that allow transfers between equipment in a shared environment, or allow sharing of equipment, when processing multiple patient samples. For a particular patient, multiple cassette streams of the same type may be run in parallel, the total number of streams (corresponding to clonal populations produced) may be set according to patient demographics or health data that is indicative of reprogramming yields, and/or by the requirements of a downstream differentiation process. For example, some processes are very low yield per iPSC clone, requiring more clones for a high confidence of manufacturing a good dose, or cases in which a large number of iPSCs is needed to start the differentiation process.

Source cells 1202 may be seeded into a first cassette 1204, which may be a two-level cassette that enables liquid changes without loss of suspension cells. For example, these may be T-cells or PBMCs that have been separated from patient blood. The cells 1202 may be activated and/or expanded in the cassette 1204, with media and reagents changed as indicated by fluid flows 1206 and 1208. Additionally, reprogramming vectors, for example, Sendai vectors, may be supplied into the cassette 1204 via the cassette ports. In alternate implementations, a cassette that enables media changes and washing of residual Sendai virus post-infection while retaining cells in suspension by other means (for example, the counterflow centrifugation cassette described herein) may be used to accomplish these operations.

After activation, expansion, and reprogramming vector addition, residual vectors (e.g., residual Sendai virus) may be washed from the cassette 1204 by a fluid media change to avoid reinfection of the cells 1202. The cells 1202 may then be transferred via a fluidic management system into several new cassettes (or several separated chambers in one or more cassettes), as indicated by arrows 1210. In the example shown in FIG. 12, the fluidic management system has diluted the source cells 1202 to different levels, for two different cell seeding densities in cassettes 1212 and 1214. This may be performed for the purpose of compensating for large patient-to-patient variations in reprogramming efficiency, and a desire for a certain range of unique clones emerging in the ultimate source cassette. With two or more seeding densities, the probability of a starting point with the correct range of emerging clones is increased. During the iPSC colony emergence phase, non-reprogrammed cells, differentiated cells, and reprogrammed cells may be imaged to track progress of iPSC colony formation and clone establishment, and cells may be removed using a cell removal tool (CRT) to manage density or remove cells that are not likely to result in iPSCs.

In the example shown in FIG. 12, cassette 1214 may be selected to start the clone observation and selection process, and its contents may be transferred to a clonalization, feature tracking, mini-expansion, and down-selection cassette 1216. In some implementations, a single cassette may perform the steps associated with cassettes 1214 and 1216. Within cassette 1216, multiple colonies may be managed with imaging and a CRT to maintain healthy density and ensure clonality. They may also be expanded in a controlled manner using CRT. Their features may be tracked, and these features may be used to down-select colonies that have, for example, the highest potential to become clonal (e.g., each iPSC colony coming from one reprogrammed cell) and to be pluripotent. The down-selection decision may be made manually by a user, autonomously by a cell manufacturing platform using a CRT and machine learning models on images, or semi-autonomously (e.g., user has approval over machine learning recommendations). In some implementations, in conjunction with the CRT or by other means described herein, cellular material may be extracted from specific colonies to profile them karyotypically, genomically, or otherwise. Ultimately, a single clonal colony is selected, and all other colonies are removed using the CRT and washing. As in other steps of this process, cell media exchange may be provided through fluidic ports of the cassette. Washing steps, as required, may also be performed through these ports using liquid-only or air-liquid steps.

Material from the selected clonal colony may be harvested and transferred to a new cassette 1218 that serves as the first expansion stage. In some implementations, a single cassette may perform the steps associated with cassettes 1216 and 1218. After sufficient expansion of the clone, which may be managed as described herein using imaging and CRT, the cells may be split and transferred to two new cassettes 1220 and 1222. Additionally, some material may be extracted (as indicated by arrow 1224) for in-process quality control or characterization, for example, to measure properties of the clone prior to full expansion, or to measure progress of reprogramming vector clearance. In some implementations, expansion processing may be extended through passaging into additional cassettes, and/or de-densification of cells within cassettes using the CRT (to minimize the number of cassette-to-cassette passages required for a certain number of target cells, while achieving consistent reprogramming vector clearance) to achieve the target level of reprogramming vector clearance. In addition, media may be tested for sterility or metabolic assessments.

After expansion is completed in cassettes 1220 and 1222, the cells may be further passaged to larger expansion cassettes 1224 and 1226. During expansion in these cassettes, spent media may be sampled and accumulated (as indicated by arrow 1228) for sterility testing or metabolic assessments. In the example shown in FIG. 12, the entire clonal cell population, from multiple expansion cassettes, may then be harvested by a fluidic management system and is processed using a filtration/liquid exchange cassette 1230 that includes two fluidic channels separated by a semipermeable membrane, that acts as a flow filtration system. Such a cassette, or cassettes with similar functions, may be used to concentrate cell material, but also to enable a liquid exchange around the cells. For example, the liquid exchange may include replacing the cell growth media (or other liquid that was used to harvest cells from the expansion cassettes) with liquid designed for cryopreservation of the cells. This enables cryo prep to be included in the cassette-based process.

Finally, the cells in the cryopreservation liquid may be flowed out of the cassette 1230, as indicated by arrow 1232. The cells may be transferred into another cassette designed to hold a cryopreservation tube, such that with a simple aseptic disconnection, such as with a tube welder, the tube may be removed from the cassette carrier and placed into a controlled-rate freezer. In some implementations, in which the system is highly automated, the cassette carrier itself may carry the cells into controlled-rate freezing and/or cryo storage. In other implementations, iPSCs resulting from the process depicted in FIG. 12 may be transferred directly into downstream processes, for example, differentiation into target cells, and quality control processes.

### Systems and Methods for Manufacturing Brown/Beige Adipose Tissues

Brown adipose tissue (BAT), comprised of brown adipocytes, plays an important role in metabolic homeostasis and thermogenesis. This cell and tissue type may therefore be a potent therapeutic cell type for treating metabolic diseases, including but not limited to obesity, type 2 diabetes, type 1 diabetes, and metabolic dysfunction-associated steatohepatitis. BAT was discovered in adult humans in 2009, and since then several approaches have been attempted to derive these cells from humans. However, primary BAT (i.e., collected from donor sources) does not readily expand in vitro. Therefore, creating an unlimited supply of brown adipose tissue/brown adipocytes requires differentiating BAT from a pluripotent stem cell (PSC) source, such as induced pluripotent stem cells (iPSCs) or human embryonic stem cells.

High efficiency, scalable, and closed manufacturing of brown adipocytes are necessary to make them suitable as a therapeutic modality, as therapeutic doses could be estimated to be within the 300 million-3 billion cell range per human adult. Current differentiation protocols to culture brown adipocytes from iPSCs range in length, and involve different cytokines to drive the differentiation, generating BATs at differing efficiencies. The available BAT differentiation protocols vary widely in approach, with some utilizing overexpression of transcription factors, some utilizing growth factors and small molecules, some utilizing cytokine cocktails, some recapitulating developmental cues, and still others using serum-based spontaneous differentiation. The culture methods also vary widely, with some protocols being carried out in 2D adherent systems and others being carried out in 3D suspension systems. Many of the available protocols succeed in the generation of brown adipocyte or brown adipocyte-like cells. However, none of these protocols are being carried out in a manner compatible with the level of scalability needed to develop a cell therapy for a highly prevalent disease, such as obesity or type 2 diabetes. Many protocols are carried out in open, manual cell manufacturing platforms, are vulnerable to inter-operator variability, have not been demonstrated to be successful across many PSC lines, and result in measurable levels of unwanted cells. These characteristics make these protocols incompatible with clinical or commercial manufacturing, which requires high yield, highly scalable, highly efficient differentiation that results in a highly pure population and works consistently across many patient-derived PSC lines.

The systems and methods disclosed herein include an automated closed-loop manufacturing process to increase yield, consistency, and scalability of PSC-based brown adipocyte differentiation protocols, to increase viability for clinical and commercial use. At a high level, the automated process disclosed herein includes (1) culture of cells, (2) automated in-process data collection of the cell culture, (3) automated analysis of the collected data to characterize the cell culture, and (4) the execution of a data-based in-process decision. In some implementations, the in-process data collection may include image-based cell culture evaluation during the brown adipocyte BAT or brown-like adipose cells differentiation process. In some implementations, the automated analysis may include AI-driven decision making and in-process culture characterization of brown adipocyte BAT or brown-like adipose cells. In some implementations, the execution of in-process decisions may include removal of undesired cells in the cell culture (e.g., using a laser-based cell removal tool) for increased derivation efficiency and increased purity of cell product. In some implementations, the automated analysis may further include AI-based prediction of successful versus failing differentiation towards brown adipocyte or brown-like adipose cells to increase overall process yield, saving costs and resources. In some implementations, the culture of brown adipocyte BAT or brown-like adipose cells may be performed in closed cassette formats, which allows for high volume tissue generation needed for transplantation into human patients.

The advantages of the present implementations include, but are not limited to: increased batch yield (in-process control results in a higher fraction of cell batches meeting the required release criteria), increased high-quality cell yield/increased purity (in-process control and selective cell removal results in an increase in the fraction of cells in a batch that are of the desired cell type), increased throughput (automated methods allow more batches to be manufactured in a given unit of time without being bottlenecked by availability of skilled labor), increased consistency (automated data analysis and decision-making reduces variability associated with operator bias), reduced cost of goods (automation and compatibility with closed manufacturing systems significantly reduce cost of goods), increased scalability (automated and closed manufacturing increases throughput and reduces cost of goods, thereby increasing scale), viability for late-stage clinical production (late-stage clinical production requires 100s of batches manufactured per year, thus requiring increased scale), and viability for commercialization (commercial production requires 1000s of batches manufactured per year, thus requiring increased scale).

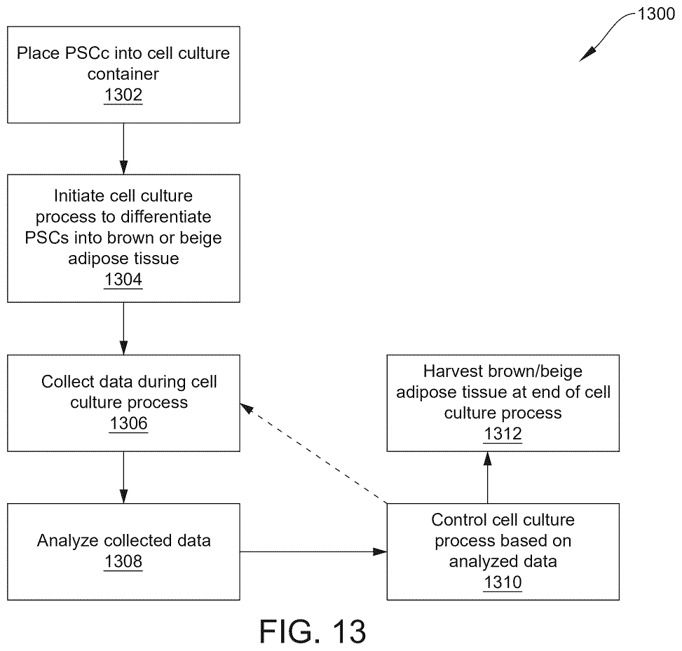

FIG. 13 is a flow chart illustrating a method 1300 of manufacturing brown or beige adipose tissue in a cell manufacturing platform in accordance with various implementations. The method 1300 may be performed by a cell manufacturing platform as described herein (e.g., the platform 100). For example, the cell manufacturing platform may utilize closed cassettes as cell culture containers for culturing brown adipocyte BAT or brown-like adipose cells. The closed cassettes may be configured to enable optical imaging and optical cell manipulation. The cell manufacturing platform may include an optical engine for capturing images of the brown/beige adipose tissue cells being cultured, a platform manager configured to analyze the images and make cell culture process decisions, and a cell removal tool (e.g., a laser system) to manipulate the brown/beige adipose tissue cells during the cell culture process.

In block 1302, PSCs (which may be induced PSCs) are placed into one or more cell culture containers (e.g., closed cassettes, well plates). In some implementations, the PSCs may be manufactured by the same cell manufacturing platform, using methods disclosed herein. In block 1304, the cell manufacturing platform may initiate a cell culture process to differentiate the PSCs into brown/beige adipose tissue cells. The cells may be cultured in a variety of formats, including using matrix-coated 2D surfaces or 3D suspension-based systems.

In block 1306, the cell manufacturing platform may be configured to collect in-process data during the cell culture process. The types of in-process data collected may include imaging and non-imaging type data. Imaging data may be of a variety of types, including but not limited to brightfield, phase-contrast, confocal, and fluorescence imaging. The imaging data may be collected by an optical engine of the cell manufacturing platform. In some implementations, the optical engine may be configured to collect images using techniques applicable for 2D monolayers, thicker 3D adherent samples, or 3D suspension samples. The in-process data may also include sensor-based measurements of metabolites, nutrient levels, dissolved gases, pH levels, and environmental parameters, such as temperature, dissolved CO₂ levels, dissolved O₂ levels, amino acid concentrations, vitamin concentrations, glucose levels, lactate levels, glutamine levels, and ammonia levels.

In block 1308, the cell manufacturing platform may be configured to analyze the collected in-process data of the cell culture. The data analysis may include a characterization made based on a single timepoint of data, or a characterization made based on a time-series-based collection of data. Examples of useful single time-point-based characterizations include: cell/colony morphology (area, shape, size, etc.), cell viability, cell density, cell location, nuclear to cytoplasm ratio, presence of subcellular organelles (e.g., mitochondria), prediction of expression levels of a critical protein, prediction of cell type (e.g., iPSC versus BAT precursor versus mature BAT cell versus contaminating cell type), cell cycle phase of a given cell, and percentage of cells in different phases. Examples of time-series-based characterizations include: cell growth, division rate, proliferation rate, cell doubling time, rate of change of the presence of a given subcellular organelle, cell doubling time, glucose consumption, lactate production, glutamine consumption, ammonia production, and rate of thermogenesis (e.g., via measuring rate of change of temperature of cell culture). The algorithms used to perform the analysis may be based on traditional computational methods (e.g., characterization of morphological features such as area of cellular region, based on traditional image analysis of brightfield images) or machine learning/artificial intelligence-based analysis methods (e.g., a deep learning algorithm is trained using pairs of brightfield images and images in which important cell characteristics are fluorescently tagged or otherwise annotated to predict expression of key proteins or presence of specific cellular organelles from brightfield images).

In block 1310, the cell manufacturing platform may be configured to control the cell culture process based on the analyzed data. For example, the data may be used to make an in-process decision that impacts the cell culture and therefore the overall manufacturing process. Examples of decisions that could be made include: removal of individual cells or regions of cells within a cell culture; new addition, increased supply, or reduced supply of a specific reagent; the advancement of the cell culture to the next stage of the differentiation/manufacturing process; transition of cell culture from 2D system to 3D system and vice versa; advancement of a batch to final quality control (QC); and/or disposal of a bad batch.

Blocks 1306-1310 may be iteratively repeated over the course of the cell culture process, thereby enabling dynamic monitoring and control of the differentiation process from PSCs to brown/beige adipose tissue cells. Once the cell culture process is complete (e.g., when a sufficient number of brown/beige adipose tissue cells meet release criteria), then the brown/beige adipose tissue cells may be harvested for cell therapy applications, as shown in block 1312. In this manner, the method 1300 provides an adaptable, scalable platform for manufacturing brown/beige adipose tissue cells for clinical and therapeutic applications.

The following are non-exhaustive examples of how the automated closed-loop cell culture process may be applied to brown/beige adipose tissue differentiation from PSCs. One aspect of the implementations disclosed herein is image-based identification and removal of unwanted cells. In some implementations, the cell manufacturing platform may perform image-based prediction of FOXC1, PAX3, and MYF5 expression to confirm the paraxial mesoderm state and remove other cells, thus enriching the BAT antecedent population and increasing purity. One approach to differentiating PSCs to BAT is through the paraxial mesoderm state, which is indicated by co-expression of FOXC1, PAX3, and MYF5. An algorithm may be trained, by pairs of brightfield images and fluorescence images in which cells in the PM state are stained for FOXC1/PAX3/MYF5, to predict expression of these markers based on images of unstained cells. This algorithm may then be used to predict regions of FOXC1+PAX3+MYF5+ cells, and the rest could be removed, for example using laser-based removal in culture, to purify the population and enrich for BAT antecedents. This process is compatible with and enables closed manufacturing systems.

In some implementations, the cell manufacturing platform may perform image-based prediction of GATA6 expression to identify brown adipocyte precursors and remove other cells, thus enriching the BAT population and increasing purity. GATA6 expression is indicative of brown adipocyte progenitors. An algorithm may be trained to predict expression of GATA6 based on in-process brightfield images of live cells. The other cells may be removed, for example using laser-based removal, to enrich for brown adipocyte progenitors and result in a higher-purity differentiated population.

In some implementations, the cell manufacturing platform may perform image-based prediction of UCP1 expression to distinguish between brown adipocytes and white adipocytes for in-process removal of white adipocytes and purification of the BAT cell population. Differentiation protocols for generating BAT from PSCs can often result in unwanted beige or white adipocytes, which do not have the same metabolic properties. For example, whereas brown adipocytes are useful for thermogenesis and energy expenditure, the most significant function of white adipocytes is energy storage. Therefore, for a cell therapy to target obesity, it would be critical to confirm the presence of brown adipocytes and smaller numbers of white adipocytes. UCP1 is expressed in brown adipocytes but not by white adipocytes. An algorithm may be trained to predict UCP1 expression based on brightfield images of unstained cells in culture. The UCP1-negative cells could then be removed, for example using laser-based cell removal. This process is compatible with and enables closed manufacturing systems.

In some implementations, the cell manufacturing platform may perform image-based prediction of cellular mitochondrial density to distinguish between brown adipocytes, beige adipocytes, and white adipocytes for in-process removal of beige adipocytes to enrich the brown adipocyte population. While beige adipocytes can perform similar thermogenesis and energy expenditure functions as brown adipocytes, they can also revert to a white adipocyte-like state, making them less desirable for a therapeutic targeting obesity and obesity-related diseases. Brown adipocytes are distinguishable from white adipocytes in that they have a much higher mitochondrial density. An algorithm may be trained, for example by pairs of brightfield and fluorescence images, in which the mitochondria are fluorescently tagged, to predict mitochondrial density based on in-process imaging of live cells. The regions of lower mitochondrial density may then be removed, for example using laser-activated cell removal, to enrich for the higher-mitochondrial-density brown adipocyte population. This process is compatible with and enables closed manufacturing systems.

Another aspect of the implementations disclosed herein is the use of image- and non-image-based characterization of cell culture composition to gate advancement to the next stage of the cell culture process. In some implementations, the cell manufacturing platform may perform image-based prediction of MSGN1 and TBX6 expression to confirm presomitic mesoderm (PSM) fate before adding growth factors to advance to the next stage of the differentiation protocol. One approach to differentiating PSCs to BAT is through the paraxial mesoderm state, which first requires differentiating to presomitic mesoderm (PSM), before further differentiating into dermomyotomal cells. The PSM state is characterized by expression of MSGN1 and TBX6. An algorithm may be trained, by pairs of brightfield images and fluorescence images in which cells in the PSM state are stained for MSGN1 and TBX6, to predict expression of these markers based on brightfield images of cells taken in-culture. This algorithm may then be used to identify which cell culture batches are in the PSM state, indicating that those batches are ready to advance to the next stage of the process in which growth factors are added to further differentiate into dermomyotomal cells.

Another aspect of the implementations disclosed herein is the use of image- and non-image-based characterization of cell culture composition to identify bad batches for disposal. In some implementations, the cell manufacturing platform may perform sensor-based in-process measurement of oxygen consumption, glucose uptake, and/or heat production for in-process assessment of cell batch functionality for the purpose of disposing of low-performing batches. BAT plays an important role in metabolic homeostasis, and some of the functions of BAT include oxygen consumption (associated with high mitochondrial density and activity), glucose uptake, and thermogenesis (conversion of chemical energy to thermal energy). In-process sensors may be used to measure these processes via change in rate of dissolved O₂, change in rate of glucose levels, and heat production, potentially in response to a stimulus. These measurements may be taken in 2D or 3D culture systems and used to assess functionality of a given manufactured BAT batch. Batches assessed to be lower-functionality may be disposed of before advancing to end-stage QC, which can be a costly and time-consuming process.

### Systems and Methods for Ex Vivo Regeneration and Rejuvenation

A lot of work is going into rejuvenation technologies with the ability to rejuvenate human (or other) cells and tissues to slow degeneration, alleviate physical and/or mental decline, and/or increase healthspan and longevity. Although preclinical work has been done in vitro and in animal models, the focus of the work is generally on in vivo rejuvenation of tissues via strategies such as partial reprogramming using Yamanaka factors. See, for example, Yücel, A. D., et al., “The long and winding road of reprogramming-induced rejuvenation.” Nature Communications 15, 1941 (2024); and Cipriano, A., et al., “Pathways and strategies for rejuvenation through epigenetic reprogramming, Nat Aging 4, 14-26 (2024), each of which is hereby incorporated by reference in its entirety.

The concept of in vivo partial reprogramming in humans is seen as very high risk because cellular reprogramming reverts cells to an intermediate and potentially ultimately pluripotent or multipotent state. In tissue rejuvenation, the goal is not to change the cell type, or to switch cells to a related stem cell or progenitor type, or to change cells to a highly defined cell type (transdifferentiation). The effects of reprogramming to the wrong cell type can be severe and even life-threatening. For example, cells reverted to a pluripotent type can form tumors as they grow and differentiate in an uncontrolled fashion. These risks, and in general the difficulty of monitoring and controlling the precise trajectory of cells during rejuvenation procedures including partial reprogramming, are slowing development of a wide range of therapies and treatments. Thus, there is a need in the art to minimize risks in the cell culture process for cell rejuvenation and regeneration cell therapy applications.

The systems and methods disclosed herein may be used for tissue rejuvenation, via ex vivo bioprocesses that enable reliable, consistent cellular rejuvenation and/or expansion, and allow optimization for high yield in such processes. Ex vivo expansion of cells and tissue is known in the art. For example, ex vivo expansion of skin cells (e.g., keratinocytes, fibroblasts) is used to enable autologous treatments of wounds, facial and other deformities, and to provide skin rejuvenation treatments by the transplantation of fibroblasts. Particularly in cases where there is “more than minimal manipulation” of the characteristics of these cells, these ex vivo processes must be carefully monitored for purity and continued cell functionality, such that transplanted cells are not damaged or carry genomic abnormalities such as altered karyotype acquired during ex vivo cell culture. The methods and systems described herein are applicable to such processes, of which ex vivo rejuvenation forms a subset.

The present implementations may be applied to a wide range of cell types, including but not limited to: fibroblasts, keratinocytes, muscle cells (including muscle stem cells, satellite cells, skeletal muscle cells, and smooth muscle cells), adipogenic cells, mesenchymal stem cells, retinal cells (including retinal ganglion cells, retinal pigment epithelial cells, and photoreceptors), neurons and neural stem cells, hepatic cells, hair follicle cells, intestinal cells and intestinal stem cells, airway epithelial cells, cardiac cells, vascular cells, olfactory cells, renal cells, chondrocytes, pancreatic cells, macrophages, endothelial cells (including but not limited to vascular endothelial cells), and other cells.

In some implementations, the systems and methods disclosed herein may be combined with transdifferentiation of cells into related cell types. In some implementations, cells may be expanded as well as rejuvenated, or expanded as part of the rejuvenation process. In some implementations, cells may be transplanted back into the patient in 2D or 3D structures, up to and including sub-organ structures or full organs. In other implementations, cells may be transplanted, for example via injection, into the existing organs to restore or enhance function and/or combat degeneration, as a result of the improved functionality of the rejuvenated cell mass and/or the trophic support or enhanced secretions of the rejuvenated cells. In some implementations, the resulting cells may be transplanted as combined cell and device, for example where cells act as an interface between the body and the device for sensing or providing signals (chemical, electrical, or other), or where cells are encapsulated in a device.

Although the examples described herein are applicable to autologous, or patient-specific, processes, the present implementations may also be applied to allogeneic cell therapies, in which donor cells are rejuvenated, expanded, and/or transdifferentiated to prepare them for transplantation. In this manner, cells from donors of any age may be rejuvenated prior to administration to patients.

Two approaches to cellular rejuvenation may be combined in the present implementations, namely cellular rejuvenation and senescent cell ablation. In cellular rejuvenation, it is important to control the cell culture process such that aberrant cell types are not present in the transplanted sample. Examples of rejuvenation protocols that may be performed in vivo but also ex vivo include telomere extension, epigenetic reprogramming, and partial reprogramming.

Telomere extension involves the use of small molecules or gene therapy to extend telomeres and restore youthful properties. For example, therapies may involve upregulation of TERT using small molecules like TA-65, use of AA Vs to deliver TERT, CRISPR activation of TERT via epigenetic modulation, or mRNA-based transient TERT expression. All of these approaches carry risks of uncontrolled outcomes, including risk of uncontrolled proliferation leading to tumor potential.

Epigenetic reprogramming involves modification of the cellular epigenome using techniques such as CRISPR-dCas9 modification of epigenetic marks to activate youthful genes or silence age-associated genes, and/or histone modification modulation including but not limited to use of HDAC inhibitors and HAT activators, DNA methylation reversal using drugs such as 5-aza-2′-deoxycytidine (Decitabine) and 5-azacytidine. All of these techniques carry risks of off-target effects that can change cell behavior, including proliferation.

Partial reprogramming involves the use of transient OSKM (Oct4, Sox2, Klf4, Myc), OSKMNL (adding LIN28 and NANOG) or OSK (omitting Myc) Yamanaka factors to epigenetically rejuvenate cells without reprogramming them (using Sendai vector, plasmids, or mRNA, for example), or chemical reprogramming techniques using small molecules to modulate epigenetic regulators. These techniques carry high risks due to potential off-target effects, and in particular due to the potential of cellular reprogramming depending on the dose and cell-specific effects, which can lead to teratoma formation upon transplantation.

The present implementations describe systems and methods for performing cellular rejuvenation processes ex vivo in a highly controlled, consistent manner that is based on continuous observation of the cell culture and its dynamics, mapping of the cell culture and its dynamics, and the removal of undesirable (or potentially undesirable) cells as identified through this mapping, by the use of automated cell removal tools (CRTs).

Specifically, states of cells or of regions of cells may be predicted via deep learning models that produce maps of cell confluence, density, phenotypic state, or as an embedding in a latent space that describes morphology, etc. These maps may be produced from label-free images and measurements of the cell culture. Furthermore, the time progression of such mapping, such as change in morphology, change in density, change in phenotype, change in embedding, can all indicate cell state. This may be combined with condition modulation techniques as described in references incorporated by reference. In some implementations, ground truth for such models may be collected via fluorescent images paired with label-free measurements. In other implementations, manual annotation may be used to create ground truth datasets. In other implementations, back-tracing a cell culture evolution and matching it to a final state that is measured via fluorescent image or other means, as described herein, may be used.

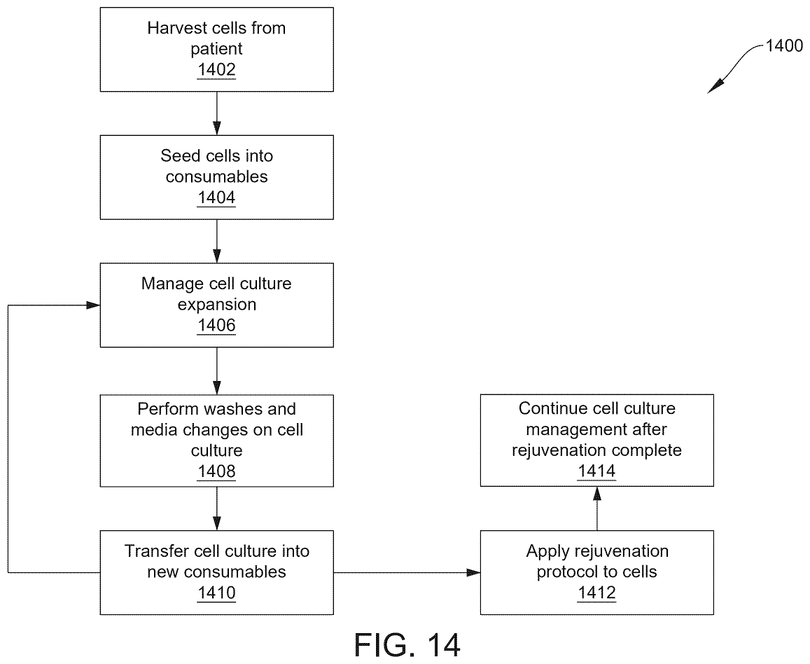

FIG. 14 is a flow chart illustrating a method 1400 of an example bioprocess for rejuvenation of specific cell types in accordance with various implementations. The method 1400 may be performed, for example, by a cell manufacturing platform that includes a series of fluidic cassettes which have a laser-absorbing coating on the cell culture surface for the purpose of removing cells via pulsed or continuous-wave laser illumination (an example CRT implementation) of this laser-absorbing surface which results in the death and/or detachment of cells (e.g., the platform 100).

In block 1402, cells may be harvested from a patient, for example by use of a biopsy from skin to extract fibroblasts (among other cells). In some implementations, the cells may be pre-treated with antibiotics to prevent proliferation of non-human cells in the cell culture process. In some implementations, the cells may be processed into single cells or cell clumps, either mechanically or chemically. For example, when harvesting fibroblast cells, the cells may be collected from small pieces of the biopsied material.

In block 1404, the cells may be seeded into a consumable for adherent cell culture. This consumable may include, but is not limited to, fluidic cell culture cassettes that enable cell culture under highly controlled conditions and with minimal contamination or cross-contamination risk. Such a consumable may be precoated with an appropriate extracellular matrix (ECM).

In block 1406, the cell manufacturing platform may be configured to perform management of the cell culture for controlled expansion. Cell culture management may include periodic imaging of the cell culture using label-free imaging and transforming the label-free images into maps of the cell culture, including one or more of: cell presence (confluence), cell density, cell morphology characteristics, cell phenotype, cell state, and/or latent space representations of the local cell culture. In some implementations, the cell manufacturing platform may utilize multiple time point images and resulting maps to compute the dynamics in each region of the consumable to capture local cell behavior, including but not limited to proliferation, morphology changes, and motility. Cell culture management further includes managing the local cell density using an algorithm and CRT, as described herein. For example, density may be managed to ensure all regions have ample growth space for expansion, so as to expand cells and also to observe the local proliferation/motility competence of cells.

Cell culture management may further include using the maps, or cell dynamics, to determine cells or regions of cells to selectively remove based on their characteristics. For example, the cell manufacturing platform may utilize a CRT to remove cells that are not the target cell type, are senescent cells (or not adequately proliferative), or may have aberrant behavior correlated with chromosomal or mutational problems based on their characteristics. Senescent cells may be identified via morphology (e.g., through deep learning models that have been trained with labeled image data so that they may pick up morphological features such as remodeled chromatin) or via their dynamics, including lower proliferation rates. In the fibroblast example, non-fibroblasts as identified by mapping may be removed with the CRT (e.g., keratinocytes may be removed from the cell culture).

In block 1408, washing and media change steps may be performed on the cell culture after cell removal by the CRT. In block 1410, for somatic cells that are sufficiently proliferative, the cells may be transferred to new consumables to provide sufficient growth area. In some implementations, the cell culture management, washing/media change, and transfer steps shown in blocks 1406-1410 may be repeated multiple times. During each repetition, active density management via CRT may be used to ensure healthy cell densities, which in turn may optimize the cell proliferation rates, which can be important to successfully apply the rejuvenation treatment such as partial reprogramming. It is known, for example, that full reprogramming of somatic cells to induced pluripotent stem cells (iPSCs) has a much higher efficiency and is sometimes only possible with cells that are actively dividing. Therefore, if portions of the cell culture are at high density and have reached a stage of contact inhibition, this can reduce the effectiveness of applied treatments.

In block 1412, a rejuvenation protocol is applied to the cells. There may be many different methods for applying a protocol. In some implementations, this may involve culturing and expanding in favorable media conditions to foster cell growth and health. In other implementations, small molecules or other compounds may be added to foster cell health, and in other implementations more extensive treatments are performed. In some implementations, the rejuvenation protocol may be performed before seeding into the initial consumable in block 1404 (for example, when a single-cell suspension is required for delivery), using through-flow electroporation or intracellular delivery of compounds via cell squeezing in a flow as examples. When the rejuvenation protocol is applied in initial seeding, lipid nanoparticle (LNP)-mRNA complexes may be added into the consumable for delivery of OSKM to the cells in culture. Such LNP-mRNA complexes may be added to media that is delivered to cells in the consumable.

In some implementations, the rejuvenation protocol process may be repeated. For example, the half-life of mRNA, which is transcribed to proteins in the cells (corresponding to OSK/OSKM, for example), is very short and so a sequence of administrations may be required. The number of repetitions, timing, and/or concentration of mRNA delivered to cells may be controlled via imaging, mapping, and cell dynamics measurements. The goal of the process is to push cells towards rejuvenation without changing cell state, and the process may be monitored by changes in cell morphology, proliferation, death, and/or motility that may appear in a fraction of the cells, or as an overall change in the mean cell characteristics. Measurements over time in response to the perturbation of mRNA addition may be useful, such that it is measured over a baseline which may be patient- or sample-dependent. In some implementations, a separate control sample may be maintained for this entire process to compare cell characteristics to a baseline over the entire process. Cells may be removed via CRT during this stage if they deviate from the expected behavior or if they approach unhealthy cell densities, for example, densities that would arrest proliferation. In some implementations, cell expansion may continue or accelerate during the application of the rejuvenation treatment. This may necessitate transfer to additional cell culture consumables and continuation of the process to yield the largest number of cells possible.

In block 1414, the cell culture process may be further managed after completion of the rejuvenation step to observe and potentially continue expansion of the cell sample. The same active density management techniques described with reference to block 1406 may be applied. The cell culture may be imaged and mapped to locate cells or regions that have characteristics or dynamics outside of the desirable range, and those cells may be removed using a CRT. Cells that may be targeted for removal include cells that appear to be of the wrong type as identified by morphology or dynamics (these may have evaded previous less stringent cell “sorting” by imaging/CRT, or result from the rejuvenation/expansion treatment), cells that appear to be senescent as identified by morphology or dynamics, and cells that appear to be reprogrammed or turned to an excessively proliferative state. These cell types are of particular concern because they may re-differentiate into the wrong cell types after transplant, or proliferate in an uncontrolled manner upon transplant. The methods described herein may be used to identify such cells or clusters of cells in situ and to remove them from the product via CRT.

Throughout the method 1400, other techniques that have been described herein may be applied, including in combination, including but not limited to various methods for measuring and tracking cells or cell regions, methods for predicting cell state, methods for managing cell density, methods for managing cell purity, etc. All the techniques described herein may be applied to standard ex vivo cell expansion processes, for both autologous as well as allogeneic processes.

In any of the steps of the method 1400, cells, cell material, or media may be extracted from the cell culture to profile the cell material. Information from such profiling (including but not limited to pH, glucose, lactate, metabolite measurements, spectral measurements, qPCR testing, proteomic testing, etc.) may be combined with image-based measurements to assess the state of the cell culture, and make better inferences at the cell/cell region level.

### Retinal Pigment Epithelial Cell Differentiation in Closed Cassette System

Dysfunction or loss of retinal pigment epithelial (RPE) cells leads to a range of conditions including age-related macular degeneration (AMD), choroideremia, and retinitis pigmentosa. Stargardt disease is a genetic disorder that leads to the degeneration of photoreceptor cells and RPEs in the macula, the central part of the retina. It results in progressive vision loss, usually starting in childhood or adolescence. In addition, the RPE layer may be damaged by physical trauma, chemical injury, or radiation injuries including excessive sun or other high-intensity (e.g., laser) light.

A number of methods have been developed to replace lost or damaged RPE cells and arrest vision loss, or even restore vision. These include autologous transplantation from the patient's own peripheral retina, transplantation from cadaveric eyes, and fetal tissue transplantation. They also include the transplantation of stem cell-derived RPEs or RPE progenitor cells. Stem cell-derived RPEs or RPE progenitors may be differentiated from embryonic stem cells (ESCs), or from iPSCs. iPSCs may be reprogrammed from a different donor (allogeneic) or from the patient's own somatic cells (autologous).

A number of challenges remain to the consistency and scalability of RPE differentiation processes, including poor differentiation efficiency or differentiation failure (often stemming from inconsistent cell culture or density in early stages of the process handling pluripotent cells), heterogeneous differentiation resulting in RPE cultures with many inclusions of non-RPE cells, heterogeneity in maturation, and the fear of residual pluripotent cells in the cell product.

Numerous methods that attempt to reduce these shortcomings have been developed. Some give vague guidance in terms of cell densities and distributions to reduce differentiation issues. Others detail one or more cell sorting or selective cell passaging steps that are inserted to remove impurities. Such sorting steps are highly non-physiological, exerting a range of stresses on cells that are naturally adherent and tightly connected to neighboring cells. These steps also result in yield losses. A number of differentiation protocols are described in the following (each of which is hereby incorporated in its entirety by reference): Leach, Lyndsay et al., “Induced Pluripotent Stem Cell-Derived Retinal Pigmented Epithelium: A Comparative Study Between Cell Lines and Differentiation Methods,” J. Ocul. Pharmacol. Ther. 2016 Jun. 1; 32(5): 317-330 (this paper describes both a spontaneous RPE differentiation as well as a directed RPE differentiation method); and Sharma, Ruchi et al., “Triphasic developmentally guided protocol to generate retinal pigment epithelium from induced pluripotent stem cells,” STAR Protocols 3, 101582, Sep. 16, 2022 (this protocol describes a different directed RPE differentiation protocol, including steps for seeding RPEs onto a scaffold for transplant as an intact sheet).

Significant advances are still required to make RPE differentiation reproducible, consistent, scalable, efficacious upon transplant, and safe. This is particularly true for autologous iPSC-derived RPEs where patient-to-patient and iPSC clone-to-clone differences mean a “one size fits all” bioprocess may result in failed or suboptimal patient doses.

The systems and methods disclosed herein include methods for RPE differentiation that are controlled via continuous cell culture imaging, computation that produces cell maps, and a CRT that selectively removes cells or cell regions from the cell culture as it proliferates and differentiates, to optimize the consistency, yield, and functionality of the resulting RPE cell product. The methods described herein are applicable to a wide range of RPE differentiation or even transdifferentiation processes including: differentiation to RPE progenitors or mature RPEs; processes yielding RPEs in suspension or RPE sheets on a scaffold or support; differentiation from allogeneic ESC, allogeneic iPSC, or autologous iPSC sources, or transdifferentiation from autologous somatic cells (including cell sources in which gene editing has been applied, for example to correct genetic issues in autologous source cells, or to improve immune compatibility in allogeneic source cells).

The methods disclosed herein may incorporate several features, in various combinations, that result in a more consistent, higher purity, higher functionality RPE cell product. For example, the methods may include source cell setup and management using imaging, computational mapping, and selective cell removal. RPE differentiation protocols are sensitive to the initial conditions established in the source cells, including overall density and density uniformity over the cell culture container. The methods may further include direction of early differentiation to enrich for RPE-destined cells using imaging, computational mapping, and selective cell removal. The methods may further include purification of mature RPEs or RPE progenitor cells by imaging, computational mapping, and selective cell removal. The methods may further include prevention or remediation of RPE “bubbling” caused by basal solute transport while maturing on a contiguous cell culture surface, by imaging, computational mapping, and selective cell removal. The methods may further include interventions to promote proper wound healing behavior by removing cells that may have formed non-proliferative/non-motile edges around holes in the RPE sheet by imaging, computational mapping, and selective cell removal. The methods may further include selective removal of lower-grade RPEs— and by virtue of wound-healing behavior, propagation of higher-grade RPEs—by imaging, computational mapping, and selective cell removal. The methods may further include completion of one or more of these process phases within a closed fluidic cassette.

In the various methods disclosed herein, imaging may comprise modalities including but not limited to label-free imaging, brightfield imaging, phase imaging, quantitative phase imaging, spectroscopic imaging, and combinations thereof. Additionally, a time series of images may be used to generate computational maps of cell features/predictions. Computational maps may be generated via heuristics based on image-extracted features, or may be generated via deep learning or other machine learning models based on a training dataset. Such models may identify present state or behavior of cells or cell regions (e.g., gaps in cell sheet, wound healing rate, pigmentation level, tightness of cell junctions, etc.) or predict future properties of cells (e.g., phagocytosis, bubbling, etc.).

Modulation of cell culture parameters may be used in conjunction with imaging or time series imaging to extract cell properties to generate computational maps. Examples of such modulation measurement methods are disclosed in U.S. patent application Ser. No. 18/972,162, titled “Systems and Methods for Analyzing and Managing Cell Culture Processes,” and filed Dec. 6, 2024, which is hereby incorporated by reference in its entirety.

Aspects of the present implementations may include various combinations of a number of base protocols/operations. An example of a base operation includes initial pluripotent cell (iPSC, ESC) seeding and density management. Another example of a base operation includes pseudo-passaging protocols but in a single cell culture container implemented using active density management, and potentially local structure management (i.e., if stripes or islands of cells are preferable). Another example of a base operation includes iPSC density control/patterning. Another example of a base operation includes ECM removal/preparation. Another example of a base operation includes active density management during initial differentiation. Another example of a base operation includes removal of improperly differentiated cells. There are various ways to detect improperly differentiated or matured RPEs, and the systems disclosed herein allow for resection of regions of the cell sheet to make space for good RPE proliferation.

Another example of a base operation includes sorting/purification at cell or region level, so that after growth has settled down, regions that may not be completely differentiated or retain some pluripotency may be removed. Characteristics that may be analyzed for cell sorting/purification include cell morphology, cell region dynamics, proliferation, mobility, rate of pigmentation increase, rate of change of cell junction contrast behavior in response to condition modulation, modulation of reagents/factors in fluid media, and spatial modulation (e.g., wound healing response after laser removal of cells).

Another example of a base operation includes bubble popping, which is the use of the CRT to burst bubbles in RPE sheets that result from basal secretions to maintain an orderly, fully imageable sheet. If the bubbles have become too large, resecting the entire (usually circular) area allows new cells to grow into the space in a 2D sheet. This may include cutting out such patches in an RPE sheet to detach, wash out, and profile live RPE cells. Another example of a base operation includes final enrichment. In cases in which the RPEs are grown on a membrane rather than on an optical film, a pulsed laser may be used to directly delete RPEs and surrounding cells, taking advantage of the high absorption of the melanosomes and using this to cause rapid bubble expansion and collapse, leading to cell death. This may also include use of large-energy pulses to remove entire cell neighborhoods when needed (including cells that have low pigmentation and cannot be targeted directly in this way). This base operation may be used in conjunction with time-series imaging to assess local RPE sheet healing capacity. Another example of a base operation includes patch selection/ranking, which is the use of image processing to identify the best patches for transplantation. All the methods and operations described herein may be performed in closed fluidic chambers (e.g., the pluggable closed cassettes disclosed herein).

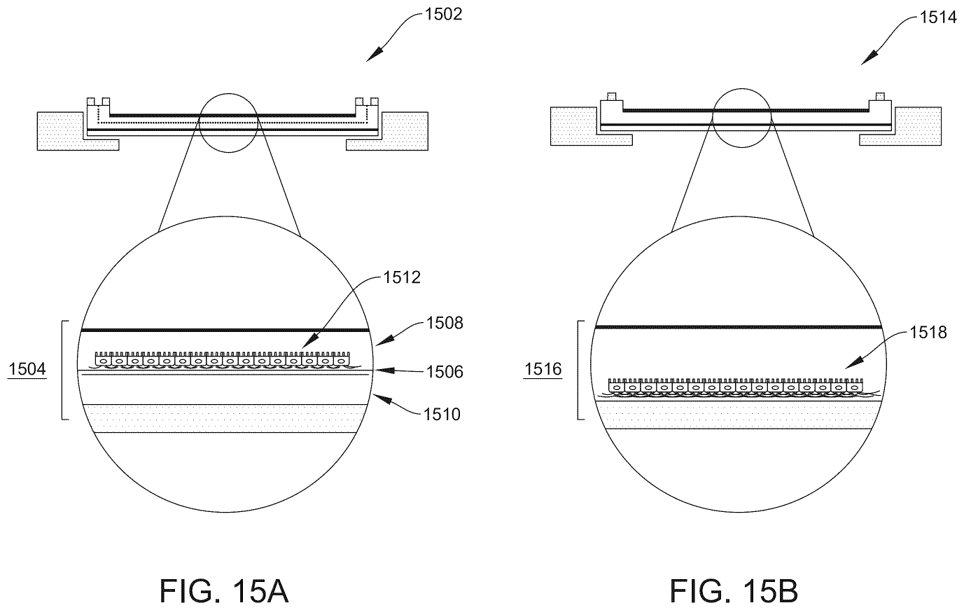

FIGS. 15A-B are diagrams illustrating closed cassettes configured to support the growth and processing of epithelial cells (e.g., RPEs) in accordance with various implementations. FIG. 15A depicts a cassette 1502 having a cell culture chamber 1504. Inside the cell culture chamber 1504 is an inner membrane 1506 that divides the chamber into two sub-chambers 1508, 1510. This configuration may allow for cell polarization dependent introduction or removal of nutrients and metabolites. The inner membrane 1506 may be composed of a single permeable material, or a bilayer which also contains a rigid supporting structure. The inner membrane 1506 may have micro-patterned openings to allow for permeation. There may also be a scaffold 1512 to structurally support the cell layer and allow for final harvesting of cell sheets. FIG. 15B depicts an alternate cassette 1514 design that does not have an inner membrane dividing its cell culture chamber 1516. The cell culture chamber 1516 may still contain a scaffold 1518 to structurally support the cell layer and allow for final harvesting of cell sheets. There may be an optical film (described herein) between the bottom surface of the cell culture chamber 1516 and the scaffold 1518. The top sides of both cassettes 1502, 1514 may be composed of a gas permeable membrane.

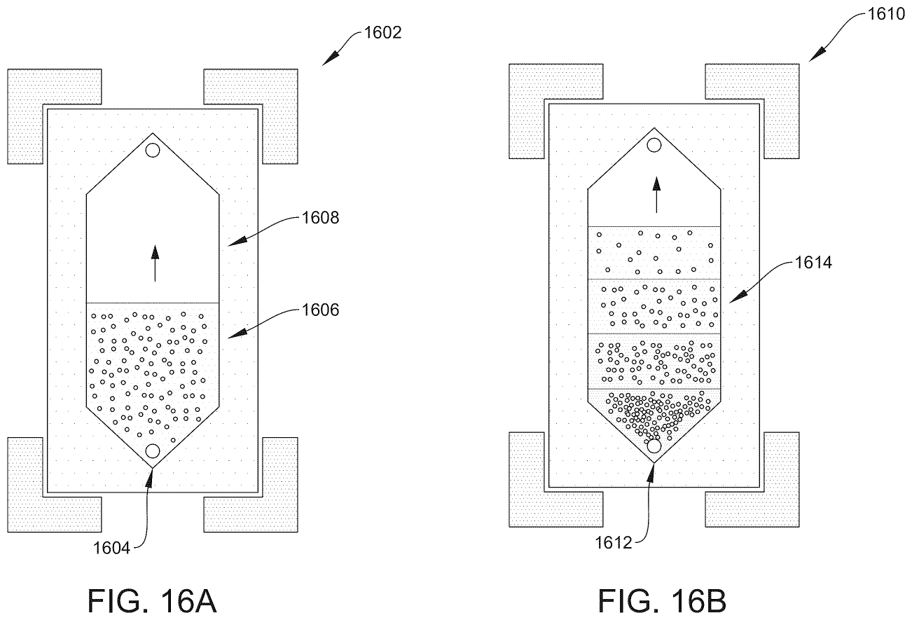

FIGS. 16A-B are diagrams depicting methods of seeding RPE cells in a closed cassette with density control in accordance with various implementations. FIG. 16A depicts a cassette 1602 oriented in a vertical direction, having a lower port 1604. The cell culture chamber of the cassette 1602 may be filled with a fluid solution 1608 having a certain density. Fluid containing RPE cells 1606 having a higher density may be introduced into the cell culture chamber via the lower port 1604, displacing the fluid solution 1608. This results in a uniform cell distribution of RPE cells in the fluid 1606. FIG. 16B depicts an alternative method of density control in RPE cell seeding, when performing an intact sheet RPE differentiation protocol. In this case, the protocol may require regions of varying cell concentration. The cassette 1610 is oriented vertically and the variation in cell density may be achieved by CRT cell patterning and removal, or by seeding subsequent solutions 1614 of increasing mass density which also contain various cell concentrations through lower port 1612.

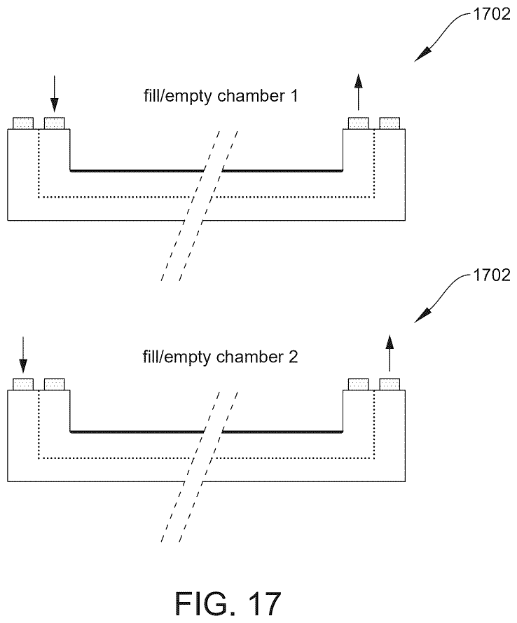

FIG. 17 is a diagram illustrating performing media changes in a closed cassette growing RPEs in accordance with various implementations. The cassette illustrated in FIG. 17 may be similar to the cassette 1502 in FIG. 15A, having the inner membrane separating the cell culture chamber into two sub-chambers. Different media solutions, having different solutes, nutrients, dissolved gases, etc., may be introduced into each sub-chamber separately, shown in media exchange operations 1702, 1704 operating on the upper sub-chamber and lower sub-chamber respectively. This allows each side of the cell layer to be exposed to different concentrations of various fluid components, which may be necessary due to the polarized nature of the cells.

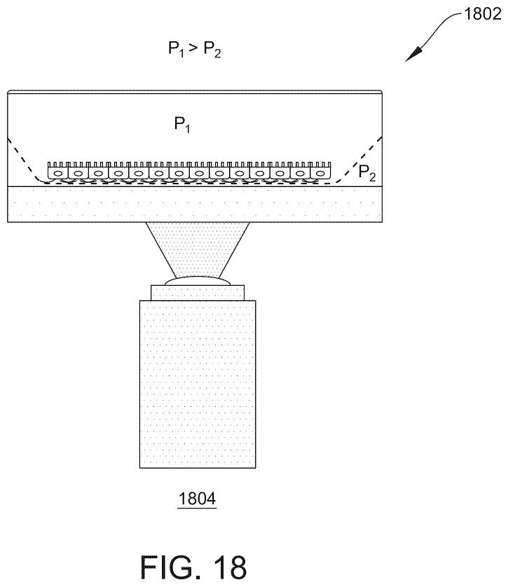

FIG. 18 is a diagram illustrating imaging of a closed cassette 1802 containing RPEs in accordance with various implementations. The imaging may be performed by an optical engine 1804 as described herein. In the implementation of a split chamber cassette (e.g., cassette 1502 in FIG. 15A) in which the inner membrane does not include a rigid supporting substrate as illustrated in FIG. 18, a pressure differential (P₁, P₂ in FIG. 18) may be applied such that the cell layer flexes downwards and becomes coplanar with the glass substrate, allowing for high-resolution imaging across the cell culture area.

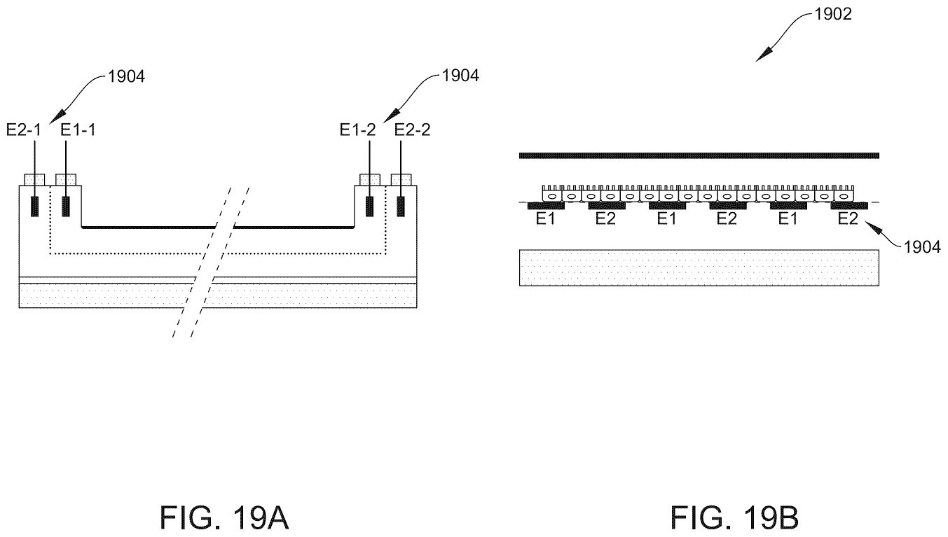

FIGS. 19A-B are diagrams of a cassette 1902 configured to make trans-epithelial electrical resistance measurements of RPE cells in accordance with various implementations. The cassette 1902 may include integrated electrodes 1904 (labeled E1-1, E1-2, E2-1, and E2-2) configured to measure trans-epithelial electrical resistance (TER). The electrodes 1904 may be near the fluidic inlets and outlets of the cassette as shown in FIG. 19A, or may be patterned along the inner membrane in an interdigitated arrangement as shown in FIG. 19B (in the case of a split chamber cassette illustrated in FIG. 15A). In the implementation illustrated in FIG. 19B, interdigitated pairs may be created such that different areas of the total cell culture are addressed, rather than measuring the sheet as a whole.

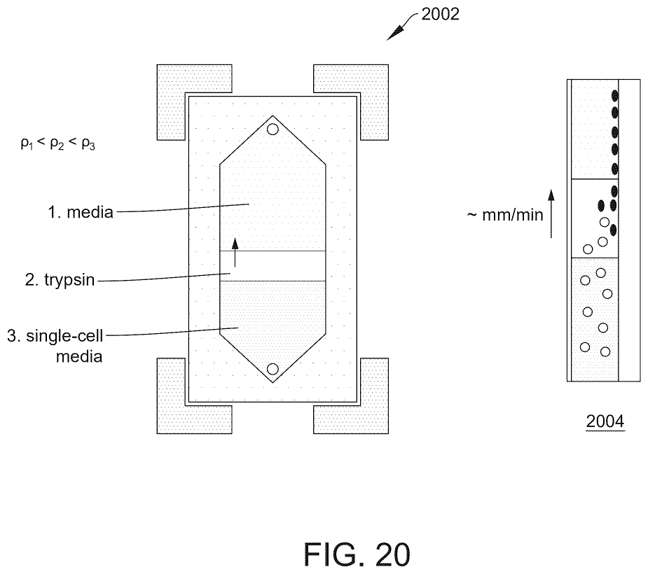

FIG. 20 is a diagram illustrating harvesting of suspension RPE cells from a cassette in accordance with various implementations. In the case in which an adherent cell culture needs to be lifted into a single cell suspension, an enzymatic or similar lifting reagent/buffer such as Trypsin may be used. However, to avoid subsequent centrifugation, it may be desirable to end up with single cells suspended in cell culture media rather than lifting buffer. To implement this, a series of different density liquids may be used in a vertically oriented cassette 2002 to achieve the right lifting time and final suspension solution. First, a thin slug of Trypsin (for example), which must be more dense than the media it is to displace, is swept up across the cell layer, slow enough to ensure full lifting during the course of exposure. Then, as the cells detach into solution, they settle downwards into an even denser solution of single-cell supporting media (shown in profile view 2004 of the cassette). The method may be used for cell harvest of many cell types within a fluidic cell culture cassette.

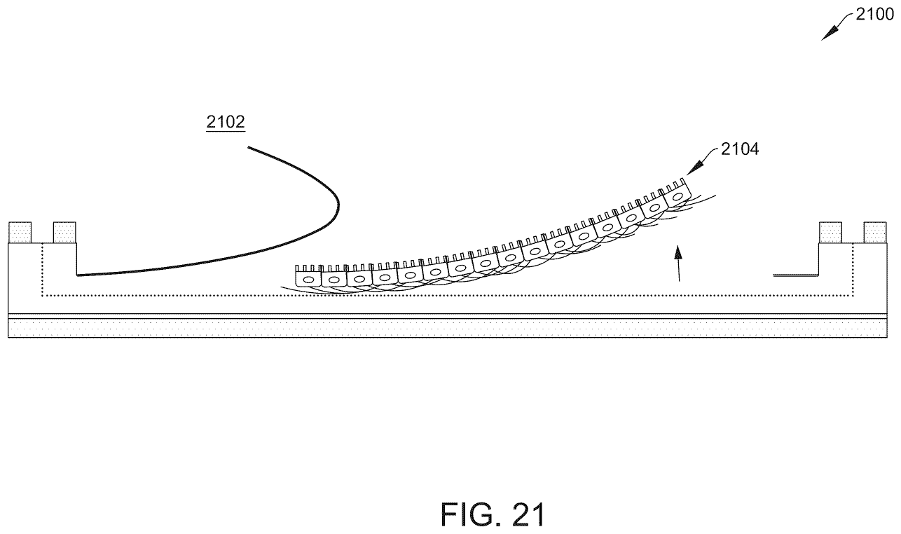

FIG. 21 is a diagram illustrating harvesting of RPE cell sheets from a cassette 2100 in accordance with various implementations. The cassette 2100 may be configured to facilitate deconstruction at the final harvesting step of the RPE cell bioprocess. In the implementation shown in FIG. 21, after removal of a top permeable roof layer 2102 of the cassette 2100, a continuous sheet of cells on a scaffold 2104 may be removed and used for subsequent steps of the end-use product. The scaffold 2104 and/or inner membrane (as shown in FIG. 15A) may also be configured with light-absorbing material such that a laser or other CRT may be used to dice the sheet into various shapes and sizes prior to or after removal.

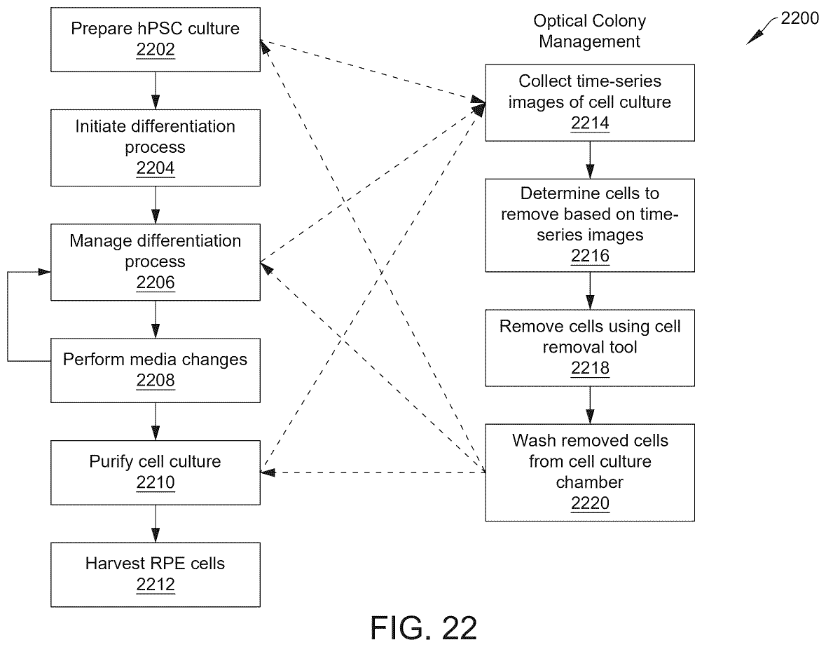

FIG. 22 is a flow chart illustrating a method 2200 of manufacturing retinal pigment epithelial tissue in a cell manufacturing platform (for example, the platform 100 in FIG. 1) in accordance with various implementations. The method 2200 utilizes optical colony management, for example laser colony management, in several steps. Optical colony management, which has been described throughout the disclosure, may include the following steps. In block 2214, the platform may collect time-series images of a cell culture (e.g., using an optical engine imaging cells in a closed cassette). In block 2216, a platform manager may utilize machine learning models to determine one or more cells to remove based on the time-series images. Which cells are to be removed depends on the current goals of the cell culture process occurring (for example, removing cells that have not differentiated during a differentiation process). In block 2218, the optical engine or other cell removal tool may remove the determined cells. In block 2220, the removed cells may be washed from the cell culture chamber (e.g., using fluidic flows).

In block 2202 of the method 2200, the platform may prepare a human pluripotent stem cell (hPSC) culture. This may include, for example, thawing hPSC cells if they are initially frozen and then culturing them in a closed cassette until the cell culture is ready for differentiation. This step may include the use of optical colony management to grow and maintain the hPSC cells in a healthy proliferative state before differentiation begins.

In block 2204, the platform may initiate the differentiation process to convert the hPSCs into RPEs. This may include introducing new fluid media to the cell culture that begins the differentiation process (e.g., cell media that includes the appropriate transcription factors that direct cell fate towards RPEs). In some implementations, the cell culture may be transferred into a different cell culture container (e.g., a new closed cassette) for differentiation.

In block 2206, the platform may manage the differentiation process. This may include the use of optical colony management to continuously monitor the cell culture and remove cells (e.g., hPSCs that have not differentiated properly, or to control cell culture density/confluence). In block 2208, the platform may perform media changes on the cell culture to refresh the fluid media and encourage further cell culture growth and development. The method 2200 may iteratively perform the steps illustrated by blocks 2206 and 2208 over a number of days (e.g., between 14-24 days).

In block 2210, the platform may purify the cell culture. Once the differentiated cells have matured, the cell culture may be purified to remove non-RPE cells, non-healthy cells, and other cells that are not suitable for cell therapy use. This step may utilize optical colony management to image and identify the unwanted cells to be removed. In block 2212, the platform may harvest the RPE cells for further use (e.g., freezing, implantation, further expansion, etc.). The method of harvesting may depend on the final form of the RPE cells (e.g., suspension, or cell sheet) and the implantation method that is to be used. In this manner, the method 2200 provides a way to autonomously manufacture RPE cells, and may be used in conjunction with various RPE differentiation protocols.

The method 2200 describes a general approach to RPE differentiation using the cell manufacturing platform disclosed herein. Specific differentiation protocols may be adapted to be performed on the platform using the method 2200. In one implementation, directed differentiation of hPSCs into RPEs (as disclosed in Leach, Lyndsay et al., “Induced Pluripotent Stem Cell-Derived Retinal Pigmented Epithelium: A Comparative Study Between Cell Lines and Differentiation Methods,” J. Ocul. Pharmacol. Ther. 2016 Jun. 1; 32(5): 317-330) may be performed as follows. If the process is starting from a frozen iPSC vial, preparing the hPSC culture may include thawing the hPSCs and removing the freezing media according to published methods. The hPSCs may be flowed into a cell culture container (e.g., a closed cassette) with fluid media and extracellular matrix, at densities described in the literature. Then the platform may utilize optical colony management to monitor the cell culture container via time series imaging (e.g., to determine cell detection, tracking, classification of pluripotency status) and remove cells to maintain the cell culture at a healthy proliferative state. The cells that may be removed include spontaneously differentiating cells or regions in the cell culture container which should not be transferred to the differentiation step (e.g., too dense, too sparse, colonies too small). The platform manager may identify the optimal culture state to begin differentiation, or terminate cultures before differentiation begins, if hPSC culture falls below morphology and behavior specifications.

Once the hPSC culture is ready to begin differentiation, then a density-dependent differentiation culture induction may be automatically determined, independent of human involvement, at the optimal time point at which cells are at the desired density and state, allowing consistent processing of every single sample. In one implementation, the hPSCs may remain in the original cell culture container and be patterned by the optical engine to initiate differentiation. In another implementation, the hPSCs may be passaged into another cell culture container to begin differentiation.

Once differentiation has begun, the platform may manage the differentiation process and perform media changes. From day 0 to 14, the differentiation media may be changed according to the published protocols. For example, the fluidic management system of the platform may control fluid media flow at appropriate rates equivalent to or better than manual media changes, with fresh cytokines/components to support constant cell exposure to differentiation reagents to avoid concentration dips. The platform may utilize optical colony management to identify and remove cells deviating from the proper RPE differentiation path throughout the differentiation process. In some implementations, depending on whether non-RPE regions are essential for RPE differentiation signaling, the optical engine may also remove cells lacking RPE morphology, and cells falling below certain cell parameter thresholds throughout the differentiation process.

On day 14, the platform may begin purifying the cell culture. For example, the platform may utilize optical colony management to track and assess cells via models to detect all regions lacking RPE morphology at single cell resolution throughout differentiation. Parameters such as pigmentation and hexagonal shape may impact model designation/score of evaluated cells and regions. Cells lacking RPE morphology and cells falling below threshold (designation/score) may be removed. Removal may be specific per cell or per region, rather than ‘rough scraping’, maximizing the amount of good cells left behind. This process results in only RPE-like cells in the culture dish. This method helps avoid whole-culture termination, and can increase success rates for donor/patient cells which are difficult to generate homogeneous good RPE cultures from.

Once an RPE-only cell culture has been achieved, the cells may be harvested. In one implementation, adherent RPE-like cells may be enzymatically digested using TrypLE, flowed out of the cell culture container, strained to single cells, and returned into a new cell culture container or the previous container, with redeposited ECM, at desired cell density with or without Y-27632 supplemented for 4 to 7 days. RPE cell counting in the cell culture container of origin done via image processing avoids the need to remove cells from the closed system, enables precise cell seeding at desired densities, enables consistency throughout RPE expansion and maintenance, and avoids stress on cells due to non-optimal cell densities. In an alternative implementation, cell lifting may be skipped and the cell culture may continue in the same cell culture container. Once harvested, the RPEs may be frozen according to the published protocols for further use.

In another implementation, spontaneous differentiation of hPSCs into RPEs (as disclosed in Leach, Lyndsay et al., “Induced Pluripotent Stem Cell-Derived Retinal Pigmented Epithelium: A Comparative Study Between Cell Lines and Differentiation Methods,” J. Ocul. Pharmacol. Ther. 2016 Jun. 1; 32(5): 317-330) may be performed as follows. The platform may first prepare the hPSC cell culture for 8-14 days. In one implementation, the hPSC culture may be overgrown according to the described protocol. In another implementation, the hPSCs may be cultured according to media conditions described in the protocol and manage the culture using optical colony management to achieve densities and cell states comparable to day 8-14 overgrown cultures. This may be performed via frequent whole-culture imaging and removal of portions of colonies to maximize high-density regions as achieved by overgrown cultures. Such platform-generated overgrown cultures may be configured to ensure uniform density across colonies in the culture to maximize RPE differentiation.

On day 14, the cell culture conditions may be changed to begin RPE differentiation, with media changes as described in the protocols. Optical colony management may be used to enrich regions going towards RPE-like fate and remove cells not going towards an RPE fate, allowing more room for RPE cells to proliferate, and increasing purity of RPE cultures. Early identification and removal increases efficiency of the protocol, increases yields, and expedites culture progression, allowing for more homogeneous cell signaling and communication.

Maturation of the cell culture is identified via image-guided machine learning models, and purification is initiated once maturation level is reached, rather than by following a 90-day rule of thumb. Such flexibility achieved by optical colony management allows for harvest at accurate timing, which could potentially cut out time from the original published protocol. Non-pigmented, non-RPE cells may be removed, at single cell resolution, leaving RPE cells adherent to the dish. The RPE cells may then be harvested according to the published protocols and seeded at equivalent densities in new cell culture containers for further expansion (with media changes according to the published protocols).

In another implementation, directed differentiation of hPSCs into RPE sheets (as disclosed in Sharma, Ruchi et al., “Triphasic developmentally guided protocol to generate retinal pigment epithelium from induced pluripotent stem cells,” STAR Protocols 3, 101582, Sep. 16, 2022) may be performed as follows. If the process starts from frozen hPSC vials, the vials may be thawed according to the published protocol and transferred into a cell culture container (e.g., a closed cassette).

The platform may then prepare the hPSC culture for differentiation using optical colony management to maintain hPSCs at appropriate division rates, density, and pluripotency state. Spontaneously differentiating cells may be removed as they are detected. In one implementation, the hPSC culture may be maintained in the same cell culture container for a time equivalent to three passages, described in the protocol as the minimum amount required to avoid peeling off of cells during the early stages of differentiation. In another implementation, the protocol may be followed and the culture passaged three times prior to differentiation. Media changes performed on day 1 may be done according to the published protocol.

On day 2, the differentiation process may begin. In one implementation, the hPSC cells may be transferred into a different cell culture container. Prior to this transfer, cell colonies that are too small may be removed and colony size may be made uniform using optical colony management. A lifting reagent may be used to detach the cells and seed them into a new cell culture container as described herein. Optical colony management may be used to adjust and homogenize the cell culture to achieve homogeneously sized colonies at the optimal density for differentiation, including adjustment of the distribution of the colonies. In another implementation, the hPSCs may remain in the original cell culture container and optical colony management may be used to adjust and homogenize the cell culture. Optical colony management may also be used to remove spontaneously differentiating cells.

From days 0-24, the platform may then manage the differentiation process and perform media changes according to the published protocol. Media changes may be performed at low speeds to avoid cell lifting from the cell culture surface. Image-guided machine learning may be used to assess various parameters such as cell death presence per culture used to track culture progression and correlate to protocol success (e.g., during days 13-18, typically present with high cell death indicative of enrichment of cells going in the RPE-direction); neuronal clusters presence per culture used to track culture progression and correlate to protocol success; flat areas of RPE progenitor cells, pigmentation, and neuronal islands per culture used to track culture progression; and ranking of regions within the culture considering morphology of pre-RPE cells, pigment, and prediction of RPE marker expression. Non-RPE cells (e.g., neuronal islands, or regions ranked low in consideration to RPE characteristics) may be detected and removed on day 25.

From day 25 to the first cell purification step, the platform manager may count RPE cells in preparation for the purification and seeding step. In one implementation, fluidics purification may be performed according to the published protocol, and then the culture is purified with two-step TrypLE to purify the RPEs, flow them out of the cell culture container, strain, and transfer them into new cell culture containers coated with vitronectin. In another implementation, optical colony management may be used to purify the cell culture. If the RPE culture does not have many contaminating non-RPE cells, purification may be skipped. If minimal non-RPE contaminants are present, optical colony management may be used to remove those contaminants, and optionally also remove the first layer of RPE cells (to promote RPE re-growth into removed regions), followed by washing and media change so that the cell culture stays in the same cell culture container. If the cells are being lifted, backup vials may be frozen or in-process samples of the cell culture may be taken.

From days 25-40, the cell culture media may be changed according to the published protocol. If the cell culture was moved to a new cell culture container during the first purification step, a media change may be performed exactly 48 hours after seeding (day 27). From day 40 to the second purification step, the platform manager may be configured to count RPE cells in culture, predict protein expression of cells in culture/maturation of the RPE cells (PMEL+, TYRP1+, MITF+), and identify CD24+CD56+ neural rosettes. The optical engine may be configured to remove cells that are predicted to not express those markers and/or are not sufficiently pigmented, have morphology of non-RPE cells, neural rosettes, and optionally the first layer of RPE cells (to promote RPE re-growth into removed regions). In one implementation, the remaining RPE cells may be lifted and homogeneously re-plated into another cell culture container. The cells are then incubated with TrypLE and monitored to measure readiness for harvest. The cells may then be collected and seeded into another cell culture container. In another implementation, the RPE cells may be kept in the same cell culture container if they are generally homogeneous and pure, or removed regions are not substantial, and will be repaired and filled up by proliferating RPE cells. The cell culture is maintained in the same or new cell culture container until day 42.

At day 42 and beyond, the platform manager may be configured to count RPE cells in culture, and predict protein expression of cells in culture/maturation of the RPE cells (PMEL+, TYRP1+, MITF+). The optical engine may be configured to remove cells that are predicted to not express those markers and/or are not sufficiently pigmented, or have morphology of non-RPE cells. The RPE cells may then be lifted according to the published protocol for harvesting into vials or seeded onto a membrane (e.g., the membrane 1506 in FIG. 15A). For the membrane seeding option, the membrane may be, for example, a PLGA biodegradable scaffold on top of a transwell, a synthetic parylene porous substrate directly on the bottom of the cell culture container (allows better imaging, and killing of individual cells), or a transwell membrane in the cell culture container. The pure RPEs are then flow-lifted into a cell culture container having the membrane at a density consistent with the published protocol, using the methods described with reference to FIGS. 16A-B. The cell culture chamber may be pre-filled with media. Settling of cells may be achieved by fluidic and robotic operations reaching the same outcome as waiting 10 minutes before moving the cell culture container to the incubator, as described in the published protocol. Media is changed at a frequency, rate, and amounts equivalent to or better than the 24-48 hour operations described in the published protocol. When the cell culture reaches maturation, equivalent to 10 days post-membrane seeding, media may be supplemented with PGE2 as described in the published protocol. The culture may continue with PGE2 supplement for the duration of the maturation process (4-5 weeks) or until sufficient maturation with high TER is achieved on the membrane. The media may be replaced at frequencies equivalent to or better than culture conditions achieved by the published protocol. Optical colony management may be used to control cell density and distribution on the membrane, targeting RPE cells directly via pigment rather than the surface on which they grow.

TER measurements and predictions on the membrane may be used as proxies to determine culture maturation and quality. In one implementation, the platform manager may utilize AI to predict TER at container and region level to inform maturation of culture and dictate harvest decisions. In another implementation, EVOM2 electrodes in the cell culture container may be used to make TER measurements (as illustrated in FIGS. 19A-B). Then the scaffold is harvested (e.g., using the method illustrated in FIG. 21). AI algorithms may be used to make decisions on which regions are suitable for use, and those regions of the sheet are cut out. In some implementations, by using TrypLE the culture time may be reduced and the optical engine may be used to cut out regions of interest.

### Manufacturing Systems and Methods for Cell Replacement Therapies

Parkinson's disease (PD) is the most prevalent neurodegenerative movement disorder, affecting over 6.1 million people globally as of 2016, a figure projected to rise significantly as the population ages. PD is characterized by the progressive degeneration of dopaminergic (DA) neurons in the substantia nigra and their projections to the striatum, leading to hallmark motor symptoms such as bradykinesia, rigidity, and resting tremor, as well as a wide range of non-motor symptoms that contribute to overall disability and reduced quality of life. Although current pharmacological and surgical treatments such as levodopa, dopamine agonists, deep brain stimulation, and focused ultrasound can offer symptomatic relief, they do not halt disease progression and often lead to complications like motor fluctuations and dyskinesias over time. This highlights the urgent need for disease-modifying therapies that address the underlying neurodegeneration in PD.

Cell replacement therapy (CRT) has emerged as a promising strategy to restore lost DA neurons and re-establish dopaminergic signaling in the striatum. Early efforts using human fetal ventral mesencephalon (hfVM) grafts showed the ability to synthesize dopamine and improve motor symptoms, but results from double-blind, placebo-controlled trials were inconsistent and complicated by ethical issues, limited tissue availability, and graft-induced dyskinesias. These challenges prompted a shift toward using more standardized and scalable sources of DA neurons derived from pluripotent stem cells, including human embryonic stem cells (hESCs) and hiPSCs. Both allogeneic (e.g., hESC-derived) and autologous (e.g., patient-specific hiPSC-derived) approaches are under investigation. Multiple protocols have been developed for deriving the transplanted cells.

For example, researchers at Mass General Brigham and McLean Hospital have developed a clinical-grade human induced pluripotent stem cell (hiPSC) differentiation protocol into midbrain dopaminergic cells (mDAC) for an autologous cell replacement therapy clinical trial in patients with sporadic Parkinson Disease. Their cell process protocols have been described in Song, Bin et al., “Human autologous iPSC-derived dopaminergic progenitors restore motor function in Parkinson's disease models,” J. Clin. Invest. 2020, 130:904-920; and Kim. J. et al., “Spotting-based differentiation of functional dopaminergic progenitors from human pluripotent stem cells,” Nat. Protocols 2022, 17:890-909; each of which is hereby incorporated by reference in their entireties.

Common challenges with these protocols, many of which are detailed in the publications, include: variability of cell behavior in a patient- and/or iPSC-clone dependent manner that may reduce consistency or yield; the need for significant hands-on expert labor throughout the cell culture process; the challenges of an open container process, which invites contamination and cross-contamination, and scaling this process to multiple patients; the length of the process, including multiple cryopreservation and quality control steps run in series; the large number of cells required at multiple stages simply for the purpose of doing quality control assays; instability of cells resulting from multiple passaging steps, and often multiple cryopreservation and thawing cycles; the heterogeneity of the final cell product in terms of cell type and cell maturity; and the fear of residual undifferentiated or pluripotent cells in the final product that could lead to tumorigenicity post-transplantation.

The present implementations disclosed herein include a method for manufacturing mDACs for transplant that solves many of these critical issues and opens the path for autologous Parkinson's treatments at a large scale and at an affordable cost. This method is built upon systems and methods described in the following references, each of which is incorporated by reference in its entirety: U.S. patent application Ser. No. 18/972,162, entitled “Systems and Methods for Analyzing and Managing Cell Culture Processes,” filed Dec. 6, 2024; and PCT Application No. PCT/US24/58832, entitled “Systems and Methods for Analyzing and Managing Cell Culture Processes,” filed Dec. 6, 2024.

The systems and methods disclosed herein may be performed in multiple cell culture container formats, but a pluggable fluidic cassette format has a number of advantages: it reduces contamination or cross-contamination exposure; it isolates cells from sloshing that is experienced in open dishes such as petri dishes, flasks or well plates during transport, media changes, and/or imaging; it reduces or eliminates evaporation which may cause variability in cell culture conditions; it presents an ideal environment for label-free transmitted-light imaging of various types (without condensation, meniscus effects); and it allows highly-controlled fluidic operations with very uniform shear forces on the cell surface, which can be critical for certain stages of the cell culture process. Moreover, the pluggable, self-sealing fluidic cassette format ultimately opens the way for multi-patient and/or small-batch manufacturing without cross-contamination issues. Finally, the format is ideal for automation, including high-speed transport systems, because of the relative isolation of cells from mechanical forces, and because a high-humidity environment is not required to slow/stop evaporation, simplifying mechatronic and optical system design. The systems and methods may be performed in such fluidic containers either in a single-patient system, or within a multi-patient bioprocessing system with appropriate aseptic connections and processes.

At multiple phases, the bioprocess is monitored and managed via label-free imaging of 100% of the cell culture area, mapping of cell characteristics across the cell culture via AI models that convert label-free images into maps, algorithms that determine based on accumulated data and maps how to proceed with the bioprocess (e.g., media and reagent changes, passaging, gas levels, etc.), and in at least some of the steps selectively remove cells from the cell culture with an automated cell removal tool (CRT). In the case of fluidic cassette-based bioprocesses, such CRT is compatible with closed container cell removal. In some implementations, an optical bioprocess is used, in which the CRT uses a light source to remove cells. For example, the CRT may be a pulsed or modulated laser interacting with a coating on the cell culture container surface to kill and/or remove cells from the cell culture.

Throughout this example implementation, fluidic operations may be performed using an automated system that plugs into the self-sealing ports of the fluidic cassettes. Automated fluidic operations give a high degree of consistency in terms of media conditions, flow rates, shear stresses, etc. In addition, in conjunction with label-free imaging systems, they enable closed-loop control of certain fluidic operations, such as cell seeding, washing, and cell harvesting. In some implementations, the automated fluidic management system may also include integrated sensors for measuring spent media characteristics including but not limited to O₂ and CO₂ dissolved gas concentrations, pH, glucose, lactate, and other media constituents.

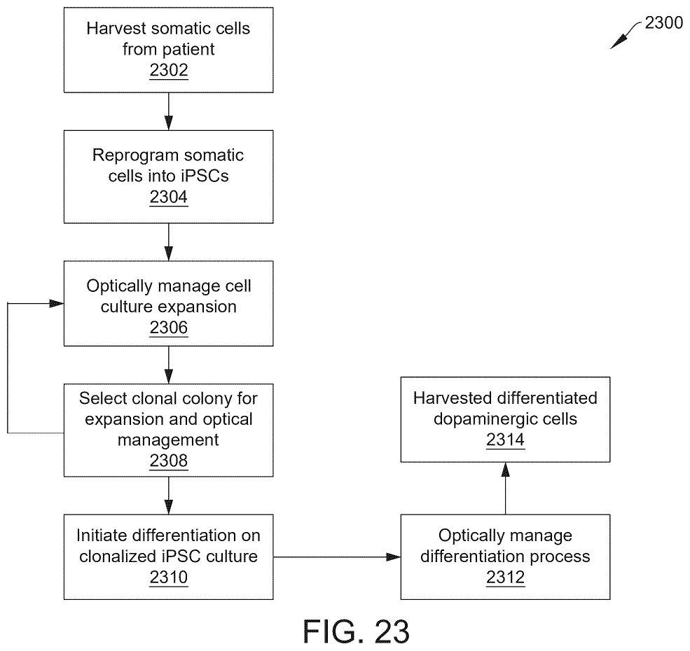

FIG. 23 is a flow chart illustrating a method 2300 of manufacturing dopaminergic cells in a cell manufacturing platform in accordance with various implementations. The method 2300 may be performed by a cell manufacturing platform as disclosed herein (e.g., the platform 100). In block 2302, somatic cells are harvested from a patient, for example blood cells, skin cells or cells derived from adipose tissue. In some implementations, the cells may be profiled via an array of assays including but not limited to DNA and RNA sequencing, methylation and/or chromatin accessibility assays, telomere assays, and functional measurements such as proliferation rate or metabolic testing. In addition, patient demographic and health data may be recorded. Both the patient data and cell data may be entered into a database where they may be used to predict various aspects of the bioprocess, including but not limited to cell expansion timing, iPSC reprogramming efficiency, likely level of iPSC defects (including but not limited to: incomplete reprogramming, karyotypic abnormalities, somatic mutations), vector clearance timing, iPSC proliferation rate, time to stabilization of iPSCs, clone differentiation efficiency, differentiation purity (into target vs off-target cell types), differentiation duration, in vitro functionality of differentiated cells, post-cry thaw cell viability, in vivo/post-transplant viability, and in vivo function. Such predictions may be updated at various points, or in some cases continually, throughout the bioprocess that transforms cells from somatic cells to iPSCs, then into differentiated cells for transplant. Predictions may be made using a variety of models informed by previous bioprocess runs and in-process and post-process assay data.

In block 2304, the harvested cells may be reprogrammed into iPSCs. Reprogramming of cells may be initiated by a variety of methods known to the art, including but not limited to episomal vectors, mRNA, viral vectors such as Sendai vector, and chemical reprogramming such as the method described in Guan, Jingyang et. al., “Chemical reprogramming of human somatic cells into pluripotent stem cells,” Nature 605, 325-331 (2022), which is included by reference in its entirety. In some implementations, the vector or factors may be added to somatic cells inside of a fluidic cassette. In alternate implementations, it may be added to cells prior to seeding in fluidic cassettes. The number of cells used, the timing of the reprogramming versus any expansion, and the amount of reprogramming vector/substance to use may be adjusted via the measurements described with reference to block 2302. The target number of iPSC clones to be manufactured, and therefore the number of cassettes that will be cultured in parallel, may also be set via these predictions.

In block 2306, the platform may manage the cell culture expansion. Management of cell culture as disclosed herein may include repeated iterations of optical imaging, data analysis/ML predictions based on the images, and selective cell removal using a CRT. As colonies resembling early iPSCs emerge in the cassette, these are mapped via label-free imaging and AI-based cell mapping models. Cell maps may be used to guide the cell removal tool (CRT) to remove regions of cells not undergoing reprogramming, remove any cells in “no-go” regions of the cassette (for example, near edges of cassette, or near fluidic inlets/outlets), to remove emerging colonies that are predicted not to result in healthy iPSCs, and/or to selectively remove colonies to achieve a target colony density within the cassette. This enables the process to accommodate a wide range of reprogramming efficiencies, while ultimately ensuring well-spaced colonies for clonalization, observation, maturation, and ultimately down-selection to a specific clone.

Cell colonies are then further managed via mapping, steering algorithms, and CRT to maintain healthy cell density, healthy total cell count per colony, and fresh ECM for the proliferating cells. A clonalization stage may optimize for rapid clonalization of each colony via steering that promotes clonalization of colonies via CRT (for example, zig-zag patterns in colony management). Then a stabilization/clearing/observation phase may manage colonies via CRT while accumulating morphological and behavioral data. The length of the phase may be determined by predictions based on patient or input cell data, via in-process observations of colony behavior, and/or measurements of material sampled from the cassette. For example, after CRT colony management, cell debris washed out of the cassette may be profiled for the reprogramming vector. At the end of this phase, a down-selection may be made via predictive models or human selection, to a smaller number of selected clonal colonies.

After this, clonal colonies may be expanded in situ, with density management via CRT. During this in situ expansion phase, an increasing number of cells per clone is available for analysis. Such analysis may be done in situ or using removed material that is profiled, as described herein. In situ analysis may include but not be limited to morphological features of the expanding colonies, dynamic behavior of the colonies, and response of the colonies to various stimuli or modulations in conditions, for example CRT-created conditions, media conditions, or dissolved gas concentrations. The behavior of each clonal colony under various perturbations may be correlated with success rates in downstream differentiation of iPSCs, and therefore may be used in clone selection. Material may be ablated selectively via the CRT and removed for analysis including but not limited to DNA sequencing, RNA sequencing, qPCR, and karyotypic, methylation, and chromatin assays. These may be used to assess any defects in the iPSCs, but also to predict success in differentiation to the target cell type.

In block 2308, the platform may select a single clonal cell colony for further expansion. In some implementations, the steps in blocks 2306 and 2308 may be iteratively repeated until the platform manager determines that the selected clonal cell colony is ready for differentiation. Ultimately all in-process data, potentially in combination with pre-existing cell/patient data, may be used in combination to select a single clone in each cassette with the maximum chance of success for differentiation into a viable transplant dose. This clone remains in the cassette, while the others are removed via CRT. After down-selection, and potentially further CRT-based management to expand the total cell number and/or monitor and delete any undesired cells growing in the cassette, the cells may be harvested and transferred to a next cassette for expansion. In this cassette the CRT may be used to manage for healthy cell density while maximizing the number of resulting cells from the expansion, as described previously, and to remove any cells that spontaneously differentiate. During the transfer to this expansion cassette, or in the subsequent transfer, live cell material may be sampled for in-process assays to confirm that the iPSCs are high quality. For clones that are not, the process may be terminated, and information recorded in databases for further training of predictive models, while other clones in parallel cassettes are moved forward in the process. One or more expansion stages may be used in the process, potentially with the increase in the number of cassettes carrying each iPS clone, and/or an expansion in the area of each growth cassette. Each of these stages may be monitored via label-free imaging and managed via CRT to healthy cell density and removal of off-target growth.

One of the aspects of the present implementation is that it does not contain an intermediate step in which iPSCs are cryopreserved, often followed by extensive characterization of the iPSCs via a range of assays. The use of a largely autonomous, cassette-based process, along with in-process characterization of iPSCs, makes the risk-adjusted cost of simply continuing the bioprocess through differentiation of the target cells relatively low, particularly if such a process is relatively short and/or requires a reasonably low number of cells for transplant. Going directly from reprogramming into expansion and then differentiation also reduces the stress on cells of cryopreservation, thawing, and recovery. It also avoids potentially open processes where contamination or cross-contamination is a risk.

Likewise, whereas many differentiation processes begin with expansion of iPSCs into banks as cryopreserved backup material, the present implementations do not create banks. Rather than trying a single iPSC clone for differentiation, multiple clones, emerging from multiple cassette streams in the iPSC process, may be differentiated in parallel, with the minimum amount of expansion before differentiation. In such a manner, the number of passages is also kept to a minimum, reducing the stress and selective pressures on the iPSC populations. Avoidance of excessive passaging can lead to more stable, more pure cell populations for subsequent differentiation. For this reason, the present implementation optimizes each expansion step using CRT to achieve maximum expansion ratios and minimize passages, avoids cryopreservation steps that introduce additional losses, and moves directly from reprogramming into differentiation.

Similarly, consistent consumables and growth surfaces are used across the process. In the present implementations, self-sealing fluidic cassettes with a laser-absorbing coating on the cell growth surface may be used to minimize adaptation requirements, times, and losses that occur when moving from one format to another. In this bioprocess the cells may be cultured in fluidic cassettes throughout, and without a break in which they are cultured in open containers or on different (for example, standard tissue culture) surfaces. In some implementations, the ECM applied to the cassettes may be transitioned between phases.

In block 2310, the platform may initiate differentiation on the clonalized iPSC colony. In the present implementations, the iPSCs may go directly from the reprogramming process into the differentiation process. The contiguous process, besides reducing cost, time, and cell stresses, enables co-optimization of iPSC reprogramming and differentiation including but not limited to: reducing the overall duration of the process, selecting iPSC clones based on likelihood of success in differentiation and transplantation, and tuning the differentiation process timing and parameters based on the real-time observed behavior of the iPSC clone. The connection between the iPSC clone reprogramming and selection process and differentiation is the iPSC clone expansion, during which time it may be characterized by image acquisition and analysis as well as sampling measurements, as described herein. When sufficient starting cells are achieved, the iPSCs are seeded into a cassette (or cassettes) that is used for differentiation. In some cases, a single cassette may be used for the entire differentiation process duration. Multiple cassettes may be run in parallel per starting iPSC clone, depending on the number of cells needed for transplant and quality control (QC).

In block 2312, the platform may optically manage the differentiation process. Upon seeding of iPSCs into the differentiation cassette, local density may be managed via CRT. In addition, cells in keep-out regions may be removed via CRT. After proliferation commences, the iPSC population may be patterned via CRT, informed by models that have determined the optimal patterning for high differentiation efficiency and yield. This may include patterning iPSCs into a series of predetermined shapes, similar to the static, manual, open-container technique described in Kim et al. However, the iPSCs may be patterned based on a combination of the observed cell densities on the growth surface, the characteristics of the present iPS clone (including but not limited to proliferation rate, epigenetic state, patient origin, etc.), and the known patterns of differentiation as observed from prior runs. In this manner, the cell culture is shaped adaptively to maximize differentiated cell yield per cassette. For example, a model may learn that a certain perimeter-to-area ratio in iPSC colony patterns, subject to certain minimums and maximums, is ideal for differentiation, and how these parameters differ with iPSC proliferation rate, and use the CRT, in conjunction with cell proliferation, to impose such a patterning on the as-seeded population. Both the macro features (e.g., colonies, which may be simple circular shapes, but also elongated shapes, zig-zag blobs, donut-shaped regions, etc.) and the within-region features such as cell density may be controlled via growth and CRT patterning of cells and ECM, specific to the clone behavior. This also implies that the timing of the start of differentiation (by change of media/factors) is dynamically calculated according to when the starting iPSCs reach the optimal state and layout. The CRT patterning may be of the cells alone, or both the cells and the ECM present in the cassette. ECM “moats” may constrain the growth of iPSC regions and be used to tune the initial iPSC density within these regions. The “moats” may be wide enough to prevent iPSC growth through them, but allow differentiated cells to cross over to fresh ECM. ECM may be removed completely via CRT, or simply depleted to slow cell motility. As described herein, motility across depleted or removed ECM may be used as a measure of cell state during the differentiation.

Differentiation may be initiated by change of media with the addition/subtraction of supplements and factors. Multiple differentiation techniques and protocols exist and are compatible with the present implementations. For example, for differentiating mDACs, floor plate induction may be initiated over days 1-5. Cell cultures may be imaged daily (or more frequently) and mapped via AI models, for characteristics including but not limited to live/dead measurement, cell density, and cell state including differentiation state. Dynamics may be observed via time-lapse imaging of the cell culture and these may also be used to generate maps. Cell density may be controlled via CRT to prevent cell detachment from overcrowding, which is a challenge for current techniques. In addition, as differentiation progresses, cell regions may be removed if they are not properly differentiating towards the target cell type. This can open up areas for properly differentiating cells to proliferate into at suitable density. For example, certain spatial features may have been required for healthy iPSC regions, and differentiation may occur preferentially around the perimeters of these original regions. After a certain point, the interior of the regions may be removed to purify the cell culture and open up additional space. Proper or improper differentiation trajectory per region may be inferred from pre-trained models as described herein.

During changes of media composition, for example in the transition from iPSC media to floor plate induction media, or then the transition to media for neural precursor induction, additional imaging timepoints may be captured to measure the dynamics of cells in finer detail. As described by reference with regards to condition modulation-based measurement, these changes in dynamics can provide additional information about cell state at a spatial level, and inform process parameter control at the global (container-level) scale using reagents, media change schedules, etc., and local (region- or cell-level) scale using the CRT. For example, after a media composition switchover, cells at a certain point in the differentiation trajectory may react by changing morphology, motility, proliferation, etc., while others may not (or may react differently). Full-container imaging and AI-based mapping can be used to detect these subtle changes and act on them to optimize overall differentiation.

Clearing of undifferentiated cells from the cell culture, which may be done at various stages of the process, is a common procedure for pluripotent-derived cell therapies. The prospect of residual undifferentiated cells in the transplanted cell product is considered a potential risk in any such therapy because of the possibility of teratoma or tumor formation that may result. Existing methods for ensuring removal of pluripotent or undifferentiated cells from differentiated cell products are reviewed in Movahed, A. Y. et al., “Elimination of tumorigenic pluripotent stem cells from their differentiated cell therapy products: An important step toward ensuring safe cell therapy,” Stem Cell Reports 2025 Jun. 7:102543, which is hereby incorporated by reference in its entirety. The methods can be summarized as follows: (1) genetic manipulation of the entire manufactured cell population to enable differential termination of residual pluripotent cells, or in the case of most suicide genes, all cells post-transplant in case of tumor emergence; (2) dissociating the entire cell population and sorting via FACS or MACS; or (3) treating the entire cell population with conditions or molecules that preferentially kill pluripotent cells. Note that in all of these cases, the entire cell population, not just the residual pluripotent cells, experiences the treatment. These treatments can have significant negative effects on the desired differentiated cells, including cell death, damage to health, or unwanted mis-differentiation, all of which may reduce the potency of the resulting cells that are transplanted into the patient. For example, in Kim et al., quercetin is applied during the neural precursor induction phase to kill undifferentiated iPSCs. While the treatment should not affect progenitors, the protocol cautions to restrict the timing of the treatment lest it destroy the differentiated cells.

Another recent review of detection methods for residual pluripotent cells is given in Watanabe, T. et al., “Evaluating teratoma formation risk of pluripotent stem cell-derived cell therapy products: a consensus recommendation from the Health and Environmental Sciences Institute's International Cell Therapy Committee,” Cytotherapy (2025), which is hereby incorporated by reference in its entirety. The review makes it clear that not only are there no methods for clearing residual pluripotent cells from cell therapy products, there are no non-destructive, in situ methods for even measuring such residual cell populations directly. All methods described for direct cell measurements rely on labels and/or destructive techniques such as qPCR, implying (a) large numbers of cells are often required solely for verifying that no residual pluripotent cells remain; and (b) the tested cells are different from the cells that will ultimately be delivered to the patient.

In the present implementations, rather than relying on post-hoc sampling measurements for detection of residual undifferentiated cells, and on in-process techniques for removing the cells that have deleterious effects on the desired cell population, the methods disclosed herein utilize label-free, in situ, continuous monitoring for residual undifferentiated cells in the transplanted population itself. Furthermore, we remove cells or regions of cells suspected to be undifferentiated selectively, without impact to the remaining cells, using the CRT. Detection of residual pluripotent cells may be done in the same manner as improperly differentiated cells, and can be done in the same process using label-free imaging and AI-based mapping. Single-timepoint detection may be done via AI models using cell and/or regional morphology, density, 3D height, etc. Using multiple timepoints to detect dynamics allows the model to incorporate motility, morphological changes, and proliferation. These may be extracted into sequences of 2D maps and then compared, or an entire multidimensional data volume (for example, multiple Z slices and multiple timepoints) processed to produce a prediction of local cell state.

Additionally, condition modulations may be applied to the cell culture and dynamics measured against these modulations. For example, media supplements may be altered as described herein. In other cases, dissolved gases (O₂, CO₂) and/or pH may be modulated to change cell behavior differentially. In other cases, fluid shear flow may be used to elicit differential behavior from cells. In other examples, compounds that affect certain types of cells may be used in small quantities. For example, quercetin may be added to the cell media at very low concentrations, and its effect on undifferentiated or poorly differentiated cells measured by a slowdown in proliferation or morphology change. Undifferentiated cells or regions identified via these measurements are then removed from the cell culture using the CRT. An important aspect of this technique is that it may be used throughout long phases of the cell differentiation process, and may be done to both remove undifferentiated cells (with very high bias), and to remove improperly or incompletely differentiating cells to ensure a high purity and potency cell product. In this manner, surveillance for undifferentiated cells has been performed long-term in the actual cells that will be transplanted, rather than a large separate sample at the end of the process, which is both only a sample, and often requires large numbers of cells to be statistically reflective of the transplanted cell product.

Many differentiation protocols incorporate passaging as a method of reducing cell density, and sometimes of preferentially re-seeding desirable cell types. Passaging is possible under the present implementations. However, it may potentially be avoided altogether via appropriate density management throughout the differentiation process, as well as removal of off-target differentiated cells or regions to increase area available for the desired cell types. In some cases, supporting cell types may be retained for a portion of the differentiation process, and then removed via CRT to purify the cell population and open up additional culture area. The avoidance, minimization, or delay of passaging reduces stresses on cells in sensitive phases of differentiation, or fragile morphology. In addition, it enables longer-duration time series tracking that increases the prediction accuracy of regional cell properties. This also allows longer-term predictions when used retrospectively to train spatio-temporal cell differentiation models such as those described herein and by reference. For this purpose, the initial density in the differentiation process may be managed to be globally low (with healthy local cell density), such that the endpoint of the differentiation is at a healthy density across the entire cassette surface. Active density management via CRT may be used throughout to prevent overcrowding that could prematurely slow differentiation or proliferation rates.

In block 2314, the platform may harvest the differentiated dopaminergic cells once the platform determines that a sufficient number of qualified and transplantable cells exist. Cell harvest may be timed from in situ cell culture observation. Often the correct level of maturity (or immaturity) of cells is very important for successful engraftment and function of cells post-transplant. The imaging-driven bioprocess described herein may drive the population in a particular cassette to the optimal point of differentiation and maturity. The cells may be allowed to continue maturing within the overall population while the platform removes cells or regions that have over-matured, until an overall optimum is achieved.

In some cassette formats, gentle cell dissociation and replacement of dissociation agent/media with cryopreservation agent may be done in the cassette, with centrifugation or filtration within the sealed cassette. Cells from multiple cassettes (but a single source iPSC clone) are then pooled prior to cryopreservation. In other formats, cells are dissociated and then put into a cryopreservation agent outside of cassettes. Fresh media from the cassettes is extracted and used to check sterility. Cells are cryopreserved in vials, with some destined for transplant, and others going to QC. The number of cells reserved for quality control must be sufficient for all standard quality tests. However, the number required for residual undifferentiated cell detection, and for cell function and purity tests may be reduced, because a large amount of data has been collected in situ that potentially enables high accuracy predictions of these within the actual transplant cell product.

While the method 2300 described herein focuses on a process for differentiation of iPSCs to mDAPs, a very similar process, with different reagents and timing, may be used to differentiate other cells, including multiple neural subtypes, in a high-yield, low-cost, scalable manner. Examples include but are not limited to GABAergic, glutamatergic, serotonergic neurons, motor neurons, oligodendrocytes, and astrocytes.

For training of models, rather than performing cell dissociation and harvest at the end of the process, the cells may be fixed in the cassette and various labelled imaging techniques may be applied to map cell phenotype spatially. For example, immunofluorescent labelling may be used. In other examples, FISH may be used to mark specific RNA molecules and thereby gene expression. This may be combined with cell extraction and single-cell RNA and/or other sequencing, either in the same cassette or other cassettes. With this information, a spatial map of cell phenotypes may be established, and through the spatial/phenotype back-tracing methods described by reference, a complete environmental/behavioral history of regions with successful and unsuccessful differentiation may be established and used to (re) train prediction and management models. For example, immunofluorescent labeling for residual pluripotent or undifferentiated cells (such as OCT4, SOX2, NANOG, TRA-1-60, SSEA or others) may be used to establish corresponding morphologies and long-term behaviors of suspect regions. In some models, such regions will reside in a specific region of embedded space (generated from morphological dynamics over time) in an AI model; then anything within a certain radius of this behavior is marked for deletion by CRT during phases of the differentiation process.

Downstream information may also be incorporated into the bioprocess management model. For example, cell viability post-thaw (prior to transplant), cell survival and proliferation post-transplant (based on imaging), and functional and safety readouts from patients may all be used to inform a quality model driving the reprogramming and differentiation process. During development, the same may be done with animal data, including imaging, functional readouts, and post-dissection tissue imaging. In this manner, the method 2300 provides an efficient, scalable, and high-quality way to obtain dopaminergic cells from somatic cells using an autonomous optical-based cell manufacturing platform.

### Other Considerations

While various implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. In addition, any combination of two or more such features, systems, aspects, articles, materials, kits, and/or methods, if such features, systems, aspects, articles, materials, kits, and/or methods are not mutually inconsistent, is included within the inventive scope of the present disclosure. Particularly, any element of the disclosure and any aspect thereof may be combined, in any order and any combination, with any other element of the disclosure and any aspect thereof.

The above-described implementations can be implemented in any of numerous ways. For example, the implementations may be implemented using hardware, software, or a combination thereof. When implemented in software, the software code can be executed on any suitable processor or collection of processors, whether provided in a single computer or distributed among multiple computers.

As used in any implementation herein, a “circuit” or “circuitry” may include, for example, singly or in any combination, hardwired circuitry, programmable circuitry, state machine circuitry, and/or firmware that stores instructions executed by programmable circuitry. An “integrated circuit” may be a digital, analog or mixed-signal semiconductor device and/or microelectronic device, such as, for example, but not limited to, a semiconductor integrated circuit chip.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smartphone or any other suitable portable or fixed electronic device. Also, a computer may have one or more input and output devices. These devices can be used, among other things, to present a user interface. Such computers may be interconnected by one or more networks in any suitable form, including a local area network or a wide area network, such as an enterprise network, an intelligent network (IN), or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Implementations of the methods described herein may be implemented using a processor and/or other programmable device. To that end, the methods described herein may be implemented on a tangible, non-transitory computer-readable medium having instructions stored thereon that, when executed by one or more processors, perform the methods. The computer-readable medium may include any type of tangible medium, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The terms “program” or “software” are used herein in a generic sense to refer to any type of computer code or set of computer-executable instructions that can be employed to program a computer or other processor to implement various aspects of implementations as discussed above. Additionally, it should be appreciated that according to one aspect, one or more computer programs that, when executed, perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various implementations. Also, data structures may be stored in computer-readable media in any suitable form.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “coupled” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals. Likewise, the terms “connected” or “coupled” as used herein in regard to mechanical or physical connections or couplings is a relative term and does not require a direct physical connection.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

It will be appreciated by those skilled in the art that any block diagrams herein represent conceptual views of illustrative circuitry embodying the principles of the disclosure. Similarly, it will be appreciated that any flow charts, flow diagrams, state transition diagrams, pseudocode, and the like represent various processes which may be substantially represented in computer-readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown. cm We claim:

1. A method of cell culture management, comprising:
(a) seeding, in a first cell culture cassette, a plurality of cell colonies adhered to a first surface of the first cell culture cassette;
(b) capturing, by an optical engine, time-series images of the plurality of cell colonies as the plurality of cell colonies expand;
(c) determining, by a platform manager, a subset of cell colonies to remove from the first surface based on the time-series images;
(d) removing, by the optical engine, the subset of cell colonies from the first surface;
(e) washing, by a fluid management system, the removed subset of cell colonies from the first cell culture cassette;
(f) repeating steps (b)-(e) for one or more iterations;
(g) harvesting, by the optical engine, a set of remaining cell colonies from the first cell culture cassette;
(h) seeding the set of remaining cell colonies into a second cell culture cassette; and
(i) repeating steps (b)-(g) for one or more iterations for the second cell culture cassette.

2. The method of claim 1, further comprising:
(j) repeating steps (h)-(i) for one or more additional cell culture cassettes.

3. The method of claim 2, further comprising harvesting cells for use in a cell therapy after the completion of step (j).

4. The method of claim 1, wherein determining the subset of cell colonies comprises applying a machine learning model to the time-series images.

5. The method of claim 1, wherein determining the subset of cell colonies comprises ranking the plurality of cell colonies according to a set of criteria and removing one or more lowest ranked cell colonies.

6. The method of claim 5, wherein the set of criteria comprises parameters corresponding to clonal colony quality.

7. The method of claim 5, wherein determining the subset of cell colonies comprises removing a portion of a highest ranked cell colony for analysis.

8. The method of claim 1, wherein determining the subset of cell colonies comprises removing cell colonies or portions of cell colonies to reduce cell confluence on the first surface.

9. The method of claim 1, wherein determining the subset of cell colonies comprises removing cell colonies or portions of cell colonies to shape remaining cell colonies in a pattern.

10. The method of claim 1, wherein harvesting the first set of remaining cell colonies comprises harvesting a single, clonal cell colony.

11. The method of claim 1, wherein harvesting the set of remaining cell colonies comprises harvesting all cells therein when cell confluence reaches a predetermined threshold.

12. The method of claim 1, wherein the set of remaining cell colonies is transferred into the second cell culture cassette as single cells, cell colonies, or cell sheets.

13. The method of claim 1, wherein the first surface comprises an optical film configured for imaging and optical removal of cells by the optical engine.

14. The method of claim 1, wherein steps (b)-(f) are performed over a period of at least 10 days.

15. The method of claim 1, wherein determining the subset of cell colonies comprises analyzing morphological characteristics of the plurality of cell colonies in the time-series images.

16. The method of claim 15, wherein the morphological characteristics include at least one of colony size, colony shape, cell density within the colony, or cell arrangement within the colony.

17. The method of claim 1, wherein the optical engine comprises a laser-based cell removal tool configured to selectively remove cells from the first surface.

18. The method of claim 1, wherein seeding the set of remaining cell colonies into the second cell culture cassette comprises dissociating the remaining cell colonies into single cells prior to seeding.

19. The method of claim 1, further comprising applying a rejuvenation protocol to the cells in the second cell culture cassette.

20. The method of claim 19, wherein the rejuvenation protocol comprises introducing factors for partial cellular reprogramming.

21. A system for cell culture management, comprising:
a first cell culture cassette, configured for a plurality of cell colonies to be seeded to and adhere to a first surface thereof;
an optical engine configured to capture time-series images of the plurality of cell colonies as the plurality of cell colonies expand;
a platform manager configured to determine a subset of cell colonies to remove from the first surface based on the time-series images, the optical engine being further configured to remove the subset of cell colonies from the first surface;
a fluid management system configured to wash the removed subset of cell colonies from the first cell culture cassette; and
a second cell culture cassette configured for a set of remaining cell colonies harvested from the first cell culture cassette to be seeded therein.

22. The system of claim 21, configure to perform a method according to any one of claims 1-20.

23. A computer-implemented method of cell culture management, comprising:
receiving time-series images of a plurality of cell colonies adhered to a first surface of a first cell culture cassette as the plurality of cell colonies expand;
determining a subset of cell colonies to remove from the first surface based on the time-series images;
sending instructions to an optical engine to remove the subset of cell colonies from the first surface;
sending instructions to a fluid management system to wash the removed subset of cell colonies from the first cell culture cassette;
sending instructions to the optical engine to harvest a set of remaining cell colonies from the first cell culture cassette; and
sending instructions to the fluid management system to seed the set of remaining cell colonies into a second cell culture cassette.

24. A computer program product for cell culture management, the computer program product comprising a computer readable storage medium having program instructions embodied therewith, the program instructions executable by a processor to cause the processor to perform a method according to claim 23.
